SEQUENCE LISTING (45 sequences)

SEQ ID NO: 1 - protein, 15 aa, Homo sapiens
GGGGSGGGGSGGGGS

SEQ ID NO: 2 - protein, 15 aa, Homo sapiens
ESGRSGGGGSGGGGS

SEQ ID NO: 3 - protein, 14 aa, Homo sapiens
EGKSSGSGSESKST

SEQ ID NO: 4 - protein, 15 aa, Homo sapiens
EGKSSGSGSESKSTQ

SEQ ID NO: 5 - protein, 14 aa, Homo sapiens
EGKSSGSGSESKVD

SEQ ID NO: 6 - protein, 14 aa, Homo sapiens
GSTSGSGKSSEGKG

SEQ ID NO: 7 - protein, 18 aa, Homo sapiens
KESGSVSSEQLAQFRSLD

SEQ ID NO: 8 - protein, 16 aa, Homo sapiens
ESGSVSSEELAFRSLD

SEQ ID NO: 9 - protein, 4 aa, Homo sapiens
KDEL

SEQ ID NO: 10 - protein, 4 aa, Homo sapiens
DDEL

SEQ ID NO: 11 - protein, 4 aa, Homo sapiens
DEEL

SEQ ID NO: 12 - protein, 4 aa, Homo sapiens
QEDL

SEQ ID NO: 13 - protein, 4 aa, Homo sapiens
RDEL

SEQ ID NO: 14 - protein, 7 aa, Homo sapiens
PKKKRKV

SEQ ID NO: 15 - protein, 7 aa, Homo sapiens
PQKKIKS

SEQ ID NO: 16 - protein, 5 aa, Homo sapiens
QPKKP

SEQ ID NO: 17 - protein, 4 aa, Homo sapiens
RKKR

SEQ ID NO: 18 - protein, 5 aa, Homo sapiens
KKKRK

SEQ ID NO: 19 - protein, 12 aa, Homo sapiens
RKKRRQRRRAHQ

SEQ ID NO: 20 - protein, 16 aa, Homo sapiens
RQARRNRRRRWRERQR

SEQ ID NO: 21 - protein, 19 aa, Homo sapiens
MPLTRRRPAASQALAPPTP

SEQ ID NO: 22 - protein, 15 aa, Homo sapiens
MDDQRDLISNNEQLP

SEQ ID NO: 23 - protein, 32 aa, Homo sapiens
  misc_feature (7)..(8): Xaa can be any naturally occurring amino acid
MLFNLRXXLNNAAFRHGHNFMVRNFRCGQPLX

SEQ ID NO: 24 - protein, 3 aa, Homo sapiens
AKL

SEQ ID NO: 25 - protein, 6 aa, Homo sapiens
SDYQRL

SEQ ID NO: 26 - protein, 8 aa, Homo sapiens
GCVCSSNP

SEQ ID NO: 27 - protein, 8 aa, Homo sapiens
GQTVTTPL

SEQ ID NO: 28 - protein, 8 aa, Homo sapiens
GQELSQHE

SEQ ID NO: 29 - protein, 8 aa, Homo sapiens
GNSPSYNP

SEQ ID NO: 30 - protein, 8 aa, Homo sapiens
GVSGSKGQ

SEQ ID NO: 31 - protein, 8 aa, Homo sapiens
GQTITTPL

SEQ ID NO: 32 - protein, 8 aa, Homo sapiens
GQTLTTPL

SEQ ID NO: 33 - protein, 8 aa, Homo sapiens
GQIFSRSA

SEQ ID NO: 34 - protein, 8 aa, Homo sapiens
GQIHGLSP

SEQ ID NO: 35 - protein, 8 aa, Homo sapiens
GARASVLS

SEQ ID NO: 36 - protein, 8 aa, Homo sapiens
GCTLSAEE

SEQ ID NO: 37 - protein, 16 aa, Homo sapiens
AAVALLPAVLLALLAP

SEQ ID NO: 38 - protein, 12 aa, Homo sapiens
AAVLLPVLLAAP

SEQ ID NO: 39 - protein, 15 aa, Homo sapiens
VTVLALGALAGVGVG

SEQ ID NO: 40 - DNA, 21 bp, Homo sapiens
atggagctccaggcagcccgc

SEQ ID NO: 41 - DNA, 23 bp, Artificial Sequence
  Description of artificial sequence  PCR primer
gccatacgggtgtgtgagccagc

SEQ ID NO: 42 - DNA, 25 bp, Artificial Sequence
  Description of artificial sequence  PCR primer
cagtggtggacctgacctgccgtct

SEQ ID NO: 43 - DNA, 27 bp, Artificial Sequence
  Description of artificial sequence  PCR primer
ctcagtgtagcccaggatgcccttgag

SEQ ID NO: 44 - DNA, 31 bp, Artificial Sequence
  Description of artificial sequence phosphorothioate-modified antisense oligonucleotides
ccagcagtaccgcttccttgccctgcggccg

SEQ ID NO: 45 - DNA, 30 bp, Artificial Sequence
  Description of artificial sequence phosphorothioate-modified antisense oligonucleotides
gccgcgtcccgttccttcaccatgacgacc

(19) United States
(12) Patent Application Publication
(10) Pub. No.: US 2005/0049176 A1
(43) Pub. Date: Mar. 3, 2005

(54) EphA2, hypoproliferative cell disorders and epithelial and endothelial reconstitution

(76) Inventors: Peter A. Kiener, Doylestown, PA (US); Michael S. Kinch, Laytonsville, MD (US); Solomon Langermann, Baltimore, MD (US)

Correspondence Address: JONES DAY, 222 EAST 41ST ST, NEW YORK, NY, 10017, US

(21) Appl. No.: 10/823,259

(22) Filed: Apr. 12, 2004

Related U.S. Application Data
(60) Provisional application No. 60/462,009, filed on Apr. 11, 2003.

(51) Int. Cl. A61K 48/00

(52) U.S. Cl. 514/2; 514/44

(57) ABSTRACT

The present invention relates to methods and compositions designed for the treatment, management, or prevention of a hypoproliferative cell disorder, especially those disorders relating to the destruction, shedding, or inadequate proliferation of epithelial and/or endothelial cells, particularly interstitial cystitis (IC) and lesions associated with inflammatory bowel disease (IBD). The methods of the invention comprise the administration of an effective amount of one or more agents that are antagonists of EphA2. In certain embodiments, the EphA2 antagonistic agent of the invention decreases EphA2-endogenous ligand binding, upregulates EphA2 gene expression and/or translation, increases EphA2 protein stability or protein accumulation, decreases EphA2 cytoplasmic tail phosphorylation, promotes EphA2 kinase activity (other than autophosphorylation or ligand-mediated EphA2 signaling), increases proliferation of EphA2 expressing cells, increases survival of EphA2 expressing cells, and/or maintains/reconstitutes epithelial and/or endothelial cell layer integrity. The invention also provides pharmaceutical compositions comprising one or more EphA2 antagonistic agents of the invention either alone or in combination with one or more other agents useful for therapy for a hypoproliferative cell disorder. Diagnostic methods and methods for screening for therapeutically useful agents are also provided.

This application claims priority to U.S. Provisional Application Ser. No. 60/462,009, filed Apr. 11, 2003, which is incorporated herein by reference in its entirety.

### 1. FIELD OF THE INVENTION

The present invention relates to methods and compositions designed for the treatment, management, or prevention of a hypoproliferative cell disorder or a disorder involving increased cell death, especially those disorders relating to the destruction, shedding, or inadequate proliferation of epithelial and/or endothelial cells, particularly interstitial cystitis (IC) and lesions associated with inflammatory bowel disease (IBD). The methods of the invention comprise the administration of an effective amount of one or more agents that are antagonists of EphA2. In certain embodiments, the EphA2 antagonistic agent of the invention upregulates EphA2 gene expression and/or translation, increases EphA2 protein stability or protein accumulation, decreases EphA2 cytoplasmic tail phosphorylation, promotes EphA2 kinase activity (other than autophosphorylation or ligand-mediated EphA2 signaling), decreases/disrupts EphA2-endogenous ligand interaction, increases proliferation of EphA2 expressing cells, increases survival of EphA2 expressing cells and/or maintains/reconstitutes cell layer integrity. In some embodiments, the EphA2 antagonist is an antibody specific for EphA2, preferably a monoclonal antibody. In other embodiments, the EphA2 antagonistic agent is a soluble endogenous ligand binding domain of EphA2. In further embodiments, the EphA2 antagonistic agent is an EphrinA1 antibody or antigen binding fragment thereof. In additional embodiments, the EphA2 antagonistic agent is a small molecule antagonist, enzymatic activity antagonist, EphrinA2 siRNA or eiRNA molecule, or EphrinA2 antisense molecule. The invention also provides pharmaceutical compositions comprising one or more agents of the invention either alone or in combination with one or more other agents useful in therapy for hypoproliferative cell disorders. Diagnostic methods and methods for screening for therapeutically useful agents are also provided.

### 2. BACKGROUND OF THE INVENTION

EphA2

EphA2 (epithelial cell kinase) is a 130 kDa member of the Eph family of receptor tyrosine kinase (Zantek N. et al, 1999, Cell Growth Differ. 10:629-38; Lindberg R. et al., 1990, Mol. Cell. Biol. 10:6316-24). The function of EphA2 is not known, but it has been suggested to regulate proliferation, differentiation, and barrier function of colonic epithelium (Rosenberg I. et al., 1997, Am. J. Physiol. 273:G824-32), vascular network assembly, endothelial migration, capillary morphogenesis, and angiogenesis (Stein E. et al., 1998, Genes Dev. 12:667-78), nervous system segmentation and axon pathfinding (Bovenkamp D. and Greer P., 2001, DNA Cell Biol. 20:203-13), tumor neovascularization (Ogawa K. et al., 2000, Oncogene 19:6043-52), and cancer metastasis (International Patent Publication Nos. WO 01/9411020, WO 96/36713, WO 01/12840, WO 01/12172).

The natural ligand of EphA2 is Ephrin A1 (Eph Nomenclature Committee, 1997, Cell 90(3):403-4; Gale, et al., 1997, Cell Tissue Res. 290(2): 227-41). The EphA2 and Ephrin A1 interaction is thought to help anchor cells on the surface of an organ and also down regulate epithelial and/or endothelial cell proliferation by decreasing EphA2 expression through EphA2 autophosphorylation (Lindberg et al., 1990, supra). Under natural conditions, the interaction helps maintain an epithelial cell barrier that protects the organ and helps regulate over proliferation and growth of epithelial cells. However, there are disease states that prevent epithelial cells from forming a protective barrier or cause the destruction and/or shedding of epithelial and/or endothelial cells and thus prevent proper healing from occurring.

Interstitial Cystitis

Interstitial cystitis (IC) is a disorder of chronic inflammation of the bladder. The exact cause of IC is unknown but it is believed to be a continuous shedding of the epithelial cells of the bladder. Studies show that the urine of IC patients contains an anti-proliferative factor (APF) that inhibits primary bladder epithelial cell proliferation, has significantly decreased levels of heparin-binding epidermal growth factor-like growth factor (HB-EGF), and increased levels of epidermal growth factor (EGF) when compared with urine from asymptomatic controls and patients with bacterial or so-called “common” cystitis (Keay S. et al., 2001, Urology 57:9-14). Other hypothesized causative factors include occult or resistant microorganisms, uroepithelial hyperpermeability, neurogenic or hormonal pathomechanisms, and mast cell activation (Oberpenning F. et al., 2002, Curr. Opin. Urol. 12:321-32).

Primary symptoms of IC are urinary frequency, urgency, pressure, tenderness, intense pain in the bladder and surrounding pelvic area, and pain during sexual intercourse. IC is far more common in women than in men. Of the more than 700,000 Americans estimated to have IC, 90 percent are women (Ratner V., 2001, World J. Urol. 19:157-9). Because IC varies in symptoms and severity, female IC patients are often misdiagnosed with bacterial cystitis and male IC patients are often misdiagnosed with prostatitis or bladder outlet obstruction.

Without a defining etiology and distinctive epidemiology, existing diagnostic tests for IC remain uncertain (Warren J. and Keay S., 2002, Curr. Opin. Urol. 12:69-74). At the present, there is neither a cure nor a standard treatment for IC. Rather, current treatment options, such as oral drugs, hydraulic bladder distention, bladder instillation, bladder wash, transcutaneous electrical nerve stimulation (TENS), and surgery, are primarily designed to alleviate the symptoms and are oftentimes either ineffective or present serious side effects (Gousse A. et al., 2000, Curr. Urol. Rep. 1: 190-8). There clearly remains a need for improved strategies of cell proliferation therapy.

Inflammatory Bowel Disease

Inflammatory bowel disease (IBD) is a term that encompasses both ulcerative colitis (inflammation of the lining of the large intestine) and Crohn's disease (inflammation of the lining and wall of the large and/or small intestine). When inflamed, the lining of the intestinal wall is red and swollen, becomes ulcerated, and bleeds. Although lesions associated with IBD can heal by themselves, most are recurrent. Chronic lesions occur in individuals with underlying diseases of various types whose medical conditions compromise the body's ability to repair injured tissue on its own (e.g., diabetes).

One type of lesion associated with IBD is an ulcer. A lesion is an open sore, an abrasion, a blister, or a shallow crater resulting from the sloughing or erosion of the top layer of epithelial cells and, sometimes, subcutaneous tissues. Although an ulcer can technically occur anywhere on the skin (e.g., a wound), the term “ulcer”, which is used loosely and interchangeably with “gastric ulcer” and “peptic ulcer”, usually refers to disorders in the upper digestive tract.

Among the many causes of ulcers are inflammation, infections (e.g., Heliobacter pylori), disorders that cause over secretion of stomach acid, emotional stress, constant pressure to the skin or muscle, etc. In particular, the pathophysiology of peptic ulcer is based on abnormalities of gastric epithelial cell function. (Kawai K. and Rokutan K., 1995, J Gastroenterol. 30(3):428-36). Common symptoms include pain and bleeding. Current treatment options include medications (e.g., proton pump blockers, antisecretory drugs, antibiotics, and antacids) and surgery which are primarily designed to relieve the symptoms and/or expedite the healing of ulcers. There clearly remains a need for improved strategies of epithelial reconstitution therapy.

### 3. SUMMARY OF THE INVENTION

EphA2 is down regulated in hypoproliferating cells and functionally altered in a number of epithelial disorders. The present inventors have found that an increase in EphA2 levels can increase the proliferation, growth, and/or survival, and/or maintain the organization of epithelial and/or endothelial cell layers. Based in part on this and other disclosures, the present invention encompasses agents and the use of agents that antagonize EphA2, i.e., decrease EphA2-endogenous ligand binding, upregulate EphA2 gene expression and/or translation, increases EphA2 protein stability or protein accumulation, decrease EphA2 cytoplasmic tail phosphorylation, promote EphA2 kinase activity (other than autophosphorylation or ligand-mediated EphA2 signaling), increase proliferation of EphA2 expressing cells, increase survival of EphA2 expressing cells, and/or maintain/reconstitute the integrity of an epithelial and/or endothelial cell layer.

The primary consequence of ligand binding is EphA2 autophosphorylation (R. A. Lindberg, et al., Molecular & Cellular Biology 10: 6316, 1990). However, unlike other receptor tyrosine kinases, EphA2 retains enzymatic activity in the absence of ligand binding or phosphotyrosine content (Zantek, et al, Cell Growth & Differentiation 10:629, 1999). The present inventors have also discovered that EphA2 promotes proliferation when unbound to ligand but inhibits proliferation when bound to its endogenous ligand, Ephrin A1. Therefore, the present invention also encompasses agents and the use of agents that decrease or disrupt EphA2 binding to its endogenous ligand.

The present invention also provides for the screening and identification of EphA2 agents that antagonize EphA2, e.g., decrease EphA2-endogenous ligand binding, upregulate EphA2 gene expression and/or translation, increases EphA2 protein stability or protein accumulation, decrease EphA2 cytoplasmic tail phosphorylation, promote EphA2 kinase activity (other than autophosphorylation or ligand-mediated EphA2 signaling), increase proliferation of EphA2 expressing cells, increase survival of EphA2 expressing cells, and/or maintain/reconstitute the integrity of an epithelial and/or endothelial cell layer. In a preferred embodiment, the EphA2 antagonistic agent of the invention is an EphA2 antibody that antagonizes EphA2, preferably a monoclonal antibody, preferable a humanized monoclonal antibody. In another preferred embodiment, the EphA2 antagonistic agent of the invention is a soluble endogenous ligand binding domain of EphA2. In further embodiments, the EphA2 antagonistic agent is an EphrinA1 antibody or antigen binding fragment. In additional embodiments, the EphA2 antagonistic agent is a small molecule antagonist, enzymatic activity antagonist, EphrinA1 siRNA or eiRNA molecule, or EphrinA1 antisense molecule. In other embodiments, the EphA2 antagonistic agent is an EphrinA2 siRNA or eiRNA molecule, or EphrinA2 antisense molecule.

In certain embodiments, the present invention relates to pharmaceutical compositions and prophylactic and therapeutic regiments designed to treat, manage, or prevent hypoproliferative cell disorders or disorders involving increased cell death, especially those disorders relating to the destruction, shedding, or inadequate proliferation of epithelial and/or endothelial cells, particularly IC and lesions associated with IBD. In some embodiments, EphA2 antagonistic agents of the invention are administered in combination with other therapeutics useful in treating such hypoproliferative cell disorders. In preferred embodiments, EphA2 antagonistic agents of the invention are administered in combination with analgesic agents, anesthetic agents, antibiotics, immunomodulatory agents or anti-urinary tract infection agents.

The invention further provides diagnostic methods using the EphA2 antagonistic agents of the invention to evaluate the efficacy of EphA2-based or non-EphA2-based treatment of hypoproliferative cell disorders, especially those disorders relating to the destruction, shedding, or inadequate proliferation of epithelial and/or endothelial cells, particularly IC and lesions associated with IBD. In general, decreased EphA2 expression is associated with increasingly severe hypoproliferation, an increased inability to repair damaged epithelial and/or endothelial cell layers, increased shedding of epithelial and/or endothelial cells and/or an increased inability to replace shedded epithelial and/or endothelial cells. Accordingly, an increase in EphA2 expression with a particular treatment indicates that the treatment is reducing the severity of hypoproliferation and/or improving epithelial and/or endothelial reconstitution. The diagnostic methods of the invention may also be used to prognose or predict hypoproliferative cell disorders or disorders involving increased cell death, especially those disorders relating to the destruction, shedding, or inadequate proliferation of epithelial and/or endothelial cells, particularly IC and lesions associated with IBD. In particular embodiments, the diagnostic methods of the invention provide methods of imaging areas of hypoproliferating or damaged epithelial and/or endothelial cells. In addition, the agents and methods of the invention may be used to diagnose, prognose or monitor therapy of (whether EphA2 or non-EphA2-based therapy) hypoproliferative cell disorders, especially those disorders relating to the destruction, shedding, or inadequate proliferation of epithelial and/or endothelial cells, particularly IC and lesions associated with IBD.

In another embodiment, kits comprising the pharmaceutical compositions or diagnostic reagents of the invention are provided.

3.1 Definitions

As used herein, the term “agent” refers to a molecule that has a desired biological effect. Agents include, but are not limited to, proteinaceous molecules, including, but not limited to, peptides, polypeptides, proteins, post-translationally modified proteins, antibodies etc., or small molecules (less than 1000 daltons), inorganic or organic compounds; or nucleic acid molecules including, but not limited to, double-stranded or single-stranded DNA, or double-stranded or single-stranded RNA (e.g., antisense, RNAi, etc.), as well as triple helix nucleic acid molecules. Agents can be derived from any known organism (including, but not limited to, animals, plants, bacteria, fungi, and protista, or viruses) or from a library of synthetic molecules. Agents that are EphA2 antagonistic agents bind to EphA2 and upregulate EphA2 gene expression and/or translation, increases EphA2 protein stability or protein accumulation, decrease EphA2 cytoplasmic tail phosphorylation, promote EphA2 kinase activity (other than autophosphorylation or ligand-mediated EphA2 signaling), decrease/disrupt EphA2-endogenous ligand interaction, increase proliferation of EphA2 expressing cells, increase survival of EphA2 expressing cells and/or maintain/reconstitute epithelial cell layer integrity. In certain embodiments, the EphA2 antagonistic agent of the invention is an EphA2 antagonist and inhibits a hypoproliferation-associated epithelial and/or endothelial cell phenotype or cell phenotype associated with increased cell death (e.g., by necrosis or apoptosis).

As used herein, the term “analog” refers to a polypeptide that possesses a similar or identical function as a particular protein (e.g., an EphA2 polypeptide), or a fragment thereof, but does not necessarily comprise a similar or identical amino acid sequence or structure of that protein or a fragment thereof. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a protein or a fragment thereof as described herein; (b) a polypeptide encoded by a nucleotide sequence that hybridizes under stringent conditions to a nucleotide sequence encoding a protein or a fragment thereof as described herein of at least 20 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a protein or a fragment thereof as described herein. A polypeptide with similar structure to a protein or a fragment thereof as described herein refers to a polypeptide that has a similar secondary, tertiary or quaternary structure of a protein or a fragment thereof as described herein. The structure of a polypeptide can be determined by methods known to those skilled in the art, including but not limited to, X-ray crystallography, nuclear magnetic resonance, and crystallographic electron microscopy. Preferably the polypeptide has EphA2 activity.

As used herein, the term “antagonist” or “antagonize” refers to any compound or action thereof, that either inhibits/decreases a molecule from binding to a natural (or endogenous) binding partner or inhibits/decreases a cellular effect that results from a molecule binding to a natural (or endogenous) binding partner. In one embodiment, an EphA2 antagonist inhibits/decreases EphA2 binding to Ephrin A1. For example, EphA2 antagonists can do one or more of the following: 1) decrease or disrupt EphA2-Ephrin A1 binding; or 2) upregulate EphA2 expression such that the amount of EphA2 on the cell surface exceeds the amount of natural ligand available for binding and thus increases the amount of unbound EphA2. In another embodiment, an EphA2 antagonist inhibits/decreases a cellular effect that results from EphA2-Ephrin A1 binding. For example, EphA2 antagonists can do one or more of the following: 1) decrease EphA2 cytoplasmic tail phosphorylation; 2) increase proliferation of EphA2 expressing cells; 3) increase survival of EphA2 expressing cells; or 4) maintain or reconstitute epithelial and/or endothelial cell layer integrity. EphA2 antagonists include, but are not limited to, biological or chemical compounds, proteins, polypeptides, peptides, antibodies, antibody fragments, nucleic acids, large or small (less than 1000 daltons) organic or inorganic molecules.

As used herein, the term “antibodies or fragments thereof that immunospecifically bind to EphA2” refers to antibodies or fragments thereof that specifically bind to an EphA2 polypeptide or a fragment of an EphA2 polypeptide and do not specifically bind to other non-EphA2 polypeptides. Preferably, antibodies or fragments that immunospecifically bind to an EphA2 polypeptide or fragment thereof do not cross-react with other antigens. Antibodies or fragments that immunospecifically bind to an EphA2 polypeptide can be identified, for example, by immunoassays or other techniques known to those of skill in the art. Antibodies of the invention include, but are not limited to, synthetic antibodies, monoclonal antibodies, recombinantly produced antibodies, multispecific antibodies (including bi-specific antibodies), human antibodies, humanized antibodies, chimeric antibodies, synthetic antibodies, intrabodies, single-chain Fvs (scFv) (e.g., including monospecific and bi-specific, etc.), Fab fragments, F(ab′) fragments, disulfide-linked Fvs (sdFv), and anti-idiotypic (anti-Id) antibodies, intrabodies, and epitope-binding fragments of any of the above. In particular, antibodies of the present invention include immunoglobulin molecules and immunologically active portions of immunoglobulin molecules, i.e., molecules that contain an antigen binding site that immunospecifically binds to an EphA2 antigen (e.g., one or more complementarity determining regions (CDRs) of an anti-EphA2 antibody). The antibodies of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. Preferably antagonistic antibodies or fragments that immunospecifically bind to an EphA2 polypeptide or fragment thereof only antagonize EphA2 and do not significantly effect other activities.

As used herein, the term “cell proliferation stimulative” refers to the ability of biological or chemical compounds, proteins, polypeptides, peptides, antibodies, antibody fragments, macromolecules, or small organic or inorganic molecules (less than 1000 daltons) to maintain, amplify, accelerate, or prolong cell proliferation, growth and/or survival in vivo or in vitro. Any method that detects cell proliferation, growth and/or survival, e.g., cell proliferation assays or epithelial barrier integrity assays, can be used to assay if an agent is a cell proliferation stimulative agent. Cell proliferation stimulative agents may also cause maintenance, regeneration, or reconstitution of epithelium when added to established colonies of hypoproliferative or damaged cells.

As used herein, the term “derivative” refers to a polypeptide that comprises an amino acid sequence of an EphA2 polypeptide, a fragment of an EphA2 polypeptide, an antibody that immunospecifically binds to an EphA2 polypeptide, or an antibody fragment that immunospecifically binds to an EphA2 polypeptide, which has been altered by the introduction of amino acid residue substitutions, deletions or additions. The term “derivative” as used herein also refers to an EphA2 polypeptide, a fragment of an EphA2 polypeptide, an antibody that immunospecifically binds to an EphA2 polypeptide, or an antibody fragment that immunospecifically binds to an EphA2 polypeptide which has been modified, i.e., by the covalent attachment of any type of molecule to the polypeptide. For example, but not by way of limitation, an EphA2 polypeptide, a fragment of an EphA2 polypeptide, an antibody, or antibody fragment may be modified, e.g., by glycosylation, acetylation, pegylation, phosphorylation, amidation, derivatization by known protecting blocking groups, proteolytic cleavage, linkage to a cellular ligand or other protein, etc. A derivative of an EphA2 polypeptide, a fragment of an EphA2 polypeptide, an antibody, or antibody fragment may be modified by chemical modifications using techniques known to those of skill in the art, including, but not limited to specific chemical cleavage, acetylation, formylation, metabolic synthesis of tunicamycin, etc. Further, a derivative of an EphA2 polypeptide, a fragment of an EphA2 polypeptide, an antibody, or antibody fragment may contain one or more non-classical amino acids. In one embodiment, a polypeptide derivative possesses a similar or identical function as an EphA2 polypeptide, a fragment of an EphA2 polypeptide, an antibody, or antibody fragment described herein. In another embodiment, a derivative of an EphA2 polypeptide, a fragment of an EphA2 polypeptide, an antibody, or antibody fragment has an altered activity when compared to an unaltered polypeptide. For example, a derivative antibody or fragment thereof can bind to its epitope more tightly or be more resistant to proteolysis.

As used herein, the term “endogenous ligand” or “natural ligand” refers to a molecule that normally binds a particular receptor in vivo. Ephrin A1 is an endogenous ligand of EphA2.

As used herein, the term “epitope” refers to a portion of an EphA2 polypeptide having antigenic or immunogenic activity in an animal, preferably in a mammal, and most preferably in a human. An epitope having immunogenic activity is a portion of an EphA2 polypeptide that elicits an antibody response in an animal. An epitope having antigenic activity is a portion of an EphA2 polypeptide to which an antibody immunospecifically binds as determined by any method well known in the art, for example, by immunoassays. Antigenic epitopes need not necessarily be immunogenic.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of an EphA2 polypeptide, a fragment of an EphA2 polypeptide, an antibody that immunospecifically binds to an EphA2 polypeptide, or an antibody fragment that immunospecifically binds to an EphA2 polypeptide which has been altered by the introduction of amino acid residue substitutions, deletions or additions. Preferably, antibody fragments are epitope-binding fragments.

As used herein, the term “humanized antibody” refers to forms of non-human (e.g., murine) antibodies, preferably chimeric antibodies, which contain minimal sequence derived from non-human immunoglobulin. For the most part, humanized antibodies are human immunoglobulins (recipient antibody) in which hypervariable region or complementarity determining (CDR) residues of the recipient are replaced by hypervariable region residues or CDR residues from an antibody from a non-human species (donor antibody) such as mouse, rat, rabbit or non-human primate having the desired specificity, affinity, and capacity. In some instances, one or more Framework Region (FR) residues of the human immunoglobulin are replaced by corresponding non-human residues or other residues based upon structural modeling, e.g., to improve affinity of the humanized antibody. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., 1986, Nature 321:522-525; Reichmann et al., 1988, Nature 332:323-329; Presta, 1992, Curr. Op. Struct. Biol. 2:593-596; and Queen et al., U.S. Pat. No. 5,585,089.

As used herein, the term “hypervariable region” refers to the amino acid residues of an antibody which are responsible for antigen binding. The hypervariable region comprises amino acid residues from a “Complementarity Determining Region” or “CDR” (i.e. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop”(i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined.

As used herein, the terms “hypoproliferative cell disorder” and “disorder involving increased cell death” refer to a disorder characterized by the destruction, shedding, or inadequate growth/proliferation or excessive cell death (e.g., apoptosis or necrosis) of a particular cell type. In preferred embodiments, the cells of the disorder are epithelial and/or endothelial cells. In further embodiments, the epithelial and/or endothelial cells are of the stomach, colon, rectum, bladder, skin, lung, pancreas, uterus, brain, gastrointestinal tract, respiratory system, circulatory system, or nervous system. Examples of such hypoproliferative cell disorders or disorders involving increased cell death include, but are not limited to, IC, chronic non-bacterial prostatitis, prostatodynia, lesions associated with IBD, ulcerative colitis, Crohn's disease, and defects in wound healing.

As used herein, the term “in combination” refers to the use of more than one prophylactic and/or therapeutic agents. The use of the term “in combination” does not restrict the order in which prophylactic and/or therapeutic agents are administered to a subject with a hypoproliferative epithelial and/or endothelial cell disorder, or disorder involving increased cell death. A first prophylactic or therapeutic agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, I week, 2 weeks, 3 weeks, 4 weeks, 5 weeks, 6 weeks, 8 weeks, or 12 weeks after) the administration of a second prophylactic or therapeutic agent to a subject which had, has, or is susceptible to a hypoproliferative epithelial and/or endothelial cell disorder. Any additional prophylactic or therapeutic agent can be administered in any order with the other additional prophylactic or therapeutic agents. In certain embodiments, EphA2 agents of the invention can be administered in combination with one or more agents (e.g., non-EphA2-based agents currently administered to treat the disorder, analgesic agents, anesthetic agents, antibiotics, immunomodulatory agents or anti-urinary tract infection agents).

As used herein, the term “low tolerance” refers to a state in which the patient suffers from side effects from treatment so that the patient does not benefit from and/or will not continue therapy because of the adverse effects and/or the harm from side effects outweighs the benefit of the treatment.

As used herein, the terms “manage”, “managing” and “management” refer to the beneficial effects that a subject derives from a prophylactic or therapeutic agent, which does not result in a cure of the disorder. In certain embodiments, a subject is administered one or more prophylactic or therapeutic agents to “manage” a disorder so as to prevent the progression or worsening of the disorder (i.e., hold disease progress).

As used herein, the term “potentiate” refers to an improvement in the efficacy of a therapeutic agent at its common or approved dose.

As used herein, the terms “prevent”, ” preventing” and “prevention” refer to the prevention of the recurrence, spread or onset of a disorder in a subject resulting from the administration of a prophylactic or therapeutic agent.

As used herein, a “prophylactically effective amount” refers to that amount of the prophylactic agent sufficient to result in the prevention of the recurrence, spread or onset of a hypoproliferative cell disorder or disorder involving increased cell death relating to the destruction and/or shedding of epithelial and/or endothelial cells, particularly IC and lesions due to IBD. A prophylactically effective amount may refer to the amount of prophylactic agent sufficient to prevent the occurrence, spread or recurrence of a hypoproliferative cell disorder or disorder involving increased cell death in a patient, including but not limited to those patients predisposed to a hypoproliferative cell disorder, for example those genetically predisposed or those having previously suffered from such a disorder. A prophylactically effective amount may also refer to the amount of the prophylactic agent that provides a prophylactic benefit in the prevention of a hypoproliferative cell disorder or disorder involving increased cell death. Further, a prophylactically effective amount with respect to a prophylactic agent of the invention means that amount of prophylactic agent alone, or in combination with one or more other agents (e.g., non-EphA2-based agents currently administered to treat the disorder, analgesic agents, anesthetic agents, antibiotics, immunomodulatory agents or anti-urinary tract infection agents) that provides a prophylactic benefit in the prevention of a hypoproliferative cell disorder. Used in connection with an amount of an EphA2 agent of the invention, the term can encompass an amount that improves overall prophylaxis or enhances the prophylactic efficacy of or synergies with another prophylactic agent.

As used herein, the term “refractory” refers to a hypoproliferative cell disorder or disorder involving increased cell death that is not responsive to one or more treatments (e.g., currently available therapies). In a certain embodiment, that a hypoproliferative cell disorder or disorder involving increased cell death is refractory to a therapy means that at least some significant portion of the symptoms associated with said disorder are not eliminated or lessened by that therapy. The determination of whether a hypoproliferative cell disorder or disorder involving increased cell death is refractory can be made either in vivo or in vitro by any method known in the art for assaying the effectiveness of treatment of a hypoproliferative cell disorder, especially IC and lesions due to IBD.

As used herein, the phrase “side effects” encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky. Examples of side effects include, but are not limited to, nausea, vomiting, anorexia, abdominal cramping, fever, pain, loss of body weight, dehydration, alopecia, dyspnea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physicians' Desk Reference (56^{th} ed., 2002).

As used herein, the term “single-chain Fv” or “sFv” refers to antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

As used herein, the term “therapeutic agent” refers to any agent that can be used in the treatment, management, prevention, or symptom reduction of a disorder associated with a hypoproliferative cell disorder relating to the destruction and/or shedding of epithelial and/or endothelial cells, particularly IC and lesions associated with IBD. In certain embodiments, the term “therapeutic agent” refers to an EphA2 antagonistic agent that inhibits a pathology-causing epithelial and/or endothelial cell phenotype. In certain embodiments, the EphA2 therapeutic agent is a monoclonal antibody or a soluble endogenous ligand binding domain of EphA2. In further embodiments, the EphA2 antagonistic agent is an Ephrin A1 antibody or antigen binding fragment. In additional embodiments, the EphA2 antagonistic agent is a small molecule antagonist, enzymatic activity antagonist, EphrinA1 siRNA or eiRNA molecule, or EphrinA1 antisense molecule. In other embodiments, the EphA2 antagonistic agent is an EphrinA2 siRNA or eiRNA molecule, or EphrinA2 antisense molecule. The term “therapeutic agent” can also refer to an agent used in anti-UTI and/or immunomodulatory therapies.

As used herein, a “therapeutically effective amount” refers to that amount of the therapeutic agent sufficient to treat, manage, or ameliorate symptoms of a disorder associated with a hypoproliferative cell disorder or disorder involving increased cell death relating to the destruction and/or shedding of epithelial and/or endothelial cells, particularly IC and lesions due to IBD, and, preferably, the amount sufficient to eliminate, modify, or control symptoms associated with such a disorder. A therapeutically effective amount may refer to the amount of therapeutic agent sufficient to delay or minimize the onset or severity of the hypoproliferative cell disorder or disorder involving increased cell death. A therapeutically effective amount may also refer to the amount of the therapeutic agent that provides a therapeutic benefit in the treatment or management of a hypoproliferative cell disorder or disorder involving increased cell death. Further, a therapeutically effective amount with respect to a therapeutic agent of the invention means that amount of therapeutic agent alone, or in combination with other therapies, that provides a therapeutic benefit in the treatment or management of a hypoproliferative cell disorder or disorder involving increased cell death. Used in connection with an amount of an EphA2 agent of the invention, the term can encompass an amount that improves overall therapy, reduces or avoids unwanted effects, or enhances the therapeutic efficacy of or synergies with another therapeutic agent.

As used herein, the terms “treat”, “treating” and “treatment” refer to the eradication, reduction or amelioration of symptoms of a disorder, particularly, the eradication, removal, modification, or control of a hypoproliferative cell disorder or disorder involving increased cell death, particularly IC or lesions associated with IBD, or regeneration and reconstitution of damaged epithelial and/or endothelial cells that results from the administration of one or more prophylactic or therapeutic agents. In certain embodiments, such terms refer to the minimizing or delay of the spread of the hypoproliferative cell disorder or disorder involving increased cell death relating to the destruction and/or shedding of epithelial and/or endothelial cells resulting from the administration of one or more prophylactic or therapeutic agents to a subject with such a disorder.

### 5. DETAILED DESCRIPTION OF THE INVENTION

EphA2 is down regulated in hypoproliferating cells and functionally altered in a number of epithelial disorders. The present inventors have found that an increase in EphA2 levels can increase the proliferation, growth and/or survival, and/or maintain the organization of cells. Based in part on this and other findings, the present invention encompasses agents and the use of agents that antagonize EphA2, e.g., decrease EphA2-endogenous ligand binding, upregulate EphA2 gene expression and/or translation, increases EphA2 protein stability or protein accumulation, decrease EphA2 cytoplasmic tail phosphorylation, promote EphA2 kinase activity (other than autophosphorylation or ligand-mediated EphA2 signaling), increase proliferation of EphA2 expressing cells, increase survival of EphA2 expressing cells, and/or maintain/reconstitute the integrity of an epithelial and/or endothelial cell layer.

The primary consequence of ligand binding is EphA2 autophosphorylation (R. A. Lindberg, et al., Molecular & Cellular Biology 10: 6316, 1990). However, unlike other receptor tyrosine kinases, EphA2 retains enzymatic activity in the absence of ligand binding or phosphotyrosine content (Zantek, et al, Cell Growth & Differentiation 10:629, 1999). The present inventors have also discovered that EphA2 promotes proliferation when unbound to ligand but inhibits proliferation when bound to its endogenous ligand, Ephrin A1. Therefore, the present invention also encompasses agents and the use of agents that decrease or disrupt EphA2 binding to its endogenous ligand.

The present invention also provides for the screening and identification of EphA2 agents that antagonize EphA2, e.g., decrease EphA2-endogenous ligand binding, upregulate EphA2 gene expression and/or translation, increases EphA2 protein stability or protein accumulation, decrease EphA2 cytoplasmic tail phosphorylation, promote EphA2 kinase activity (other than autophosphorylation or ligand-mediated EphA2 signaling), increase proliferation of EphA2 expressing cells, increase survival of EphA2 expressing cells, and/or maintain/reconstitute the integrity of an epithelial and/or endothelial cell layer. In a preferred embodiment, the EphA2 antagonistic agent of the invention is an EphA2 antibody, preferably a monoclonal antibody, preferable a humanized monoclonal antibody. In another preferred embodiment, the EphA2 antagonistic agent of the invention is a soluble endogenous ligand binding domain of EphA2. In another embodiment, the EphA2 antagonistic agent inhibits EphrinA1 expression. In a further embodiment, the EphA2 antagonistic agent is an EphrinA1 antibody or antigen binding fragment. In additional embodiments, the EphA2 antagonistic agent is a small molecule antagonist, enzymatic activity antagonist, EphrinA2 siRNA or eiRNA molecule, or EphrinA2 antisense molecule. In other embodiments, the EphA2 antagonistic agent is an EphrinA2 siRNA or eiRNA molecule, or EphrinA2 antisense molecule.

5.1 EphA2 Antagonistic Agents

As discussed above, the invention encompasses administration of antagonists that decrease EphA2-endogenous ligand binding, upregulate EphA2 gene expression and/or translation, increases EphA2 protein stability or protein accumulation, decrease EphA2 cytoplasmic tail phosphorylation, promote EphA2 kinase activity (other than autophosphorylation or ligand-mediated EphA2 signaling), increase proliferation of EphA2 expressing cells, increase survival of EphA2 expressing cells, and/or maintain/reconstitute the integrity of an epithelial and/or endothelial cell layer. Such antagonistic agents of the invention include, but are not limited to, proteinaceous molecules, including, but not limited to, peptides, polypeptides, proteins, post-translationally modified proteins, antibodies etc., or small molecules (less than 1000 daltons), inorganic or organic compounds; or nucleic acid molecules including, but not limited to, double-stranded or single-stranded DNA, or double-stranded or single-stranded RNA (e.g., antisense, mediates RNAi, etc.), as well as triple helix nucleic acid molecules.

5.2 Polypeptide Antagonistic Agents

Methods of the present invention encompasses EphA2 antagonistic agents that are polypeptides. In one embodiment, a polypeptide antagonistic agent is an EphA2 antibody or fragment thereof that immunospecifically binds EphA2 and antagonizes EphA2 (e.g., decreases EphA2-endogenous ligand binding, upregulates EphA2 gene expression and/or translation, increases EphA2 protein stability or protein accumulation, decreases EphA2 cytoplasmic tail phosphorylation, promotes EphA2 kinase activity (other than autophosphorylation or ligand-mediated EphA2 signaling), increases proliferation of EphA2 expressing cells, increases survival of EphA2 expressing cells, and/or maintains or reconstitutes the integrity of an epithelial and/or endothelial cell layer). In another embodiment, a polypeptide antagonistic agent is an EphA2 fragment that is capable of binding an EphA2 ligand (e.g., Ephrin A1) and antagonizes EphA2 (e.g., decreases EphA2-endogenous ligand binding, upregulates EphA2 gene expression and/or translation, increases EphA2 protein stability or protein accumulation, decreases EphA2 cytoplasmic tail phosphorylation, promotes EphA2 kinase activity (other than autophosphorylation or ligand-mediated EphA2 signaling), increases proliferation of Eph2 expressing cells, increases survival of EphA2 expressing cells, and/or maintains/reconstitutes the integrity of an epithelial and/or endothelial cell layer).

5.2.1 Antibodies As Polypeptide Antagonistic Agents

In one embodiment, the EphA2 antagonistic agent is an antibody, preferably a monoclonal antibody. Antibody antagonistic agents of the invention immunospecifically bind EphA2 and antagonize EphA2. In a more specific embodiment, an antibody of the invention immunospecifically binds to the extracellular domain of EphA2 (e.g., at an epitope either within or outside of the EphA2 ligand binding site) and decreases EphA2 cytoplasmic tail phosphorylation without causing EphA2 degradation. In another specific embodiment, the antibody binds to the extracellular domain of EphA2 (e.g., at an epitope either within or outside of the EphA2 ligand binding site) and inhibits or reduces the extent of EphA2-ligand interaction. In another specific embodiment, the antibody binds to the extracellular domain of EphA2 (e.g., at an epitope either within or outside of the EphA2 ligand binding site) and increases, accelerates and/or prolongs cell proliferation, growth and/or survival of an EphA2-expressing cell. In another specific embodiment, the antibody binds to the extracellular domain of EphA2 (e.g., at an epitope either within or outside of the EphA2 ligand binding site) and maintains/reconstitutes the integrity of an epithelial and/or endothelial cell layer.

Antibodies of the invention include, but are not limited to, monoclonal antibodies, synthetic antibodies, recombinantly produced antibodies, multispecific antibodies (including bi-specific antibodies), human antibodies, humanized antibodies, chimeric antibodies, synthetic antibodies, intrabodies, single-chain Fvs (scFv) (e.g., including monospecific and bi-specific, etc.), Fab fragments, F(ab′) fragments, disulfide-linked Fvs (sdFv), intrabodies, and epitope-binding fragments of any of the above. In particular, antibodies used in the methods of the present invention include immunoglobulin molecules and immunologically active portions of immunoglobulin molecules, i.e., molecules that contain at least one antigen binding site that immunospecifically binds to EphA2 and are antagonists of EphA2 (e.g., decrease EphA2-endogenous ligand binding, upregulate EphA2 gene expression and/or translation, increases EphA2 protein stability or protein accumulation, decrease EphA2 cytoplasmic tail phosphorylation, promote EphA2 kinase activity (other than autophosphorylation or ligand-mediated EphA2 signaling), increase proliferation of EphA2 expressing cells, increase survival of EphA2 expressing cells, and/or maintain/reconstitute the integrity of an epithelial and/or endothelial cell layer). The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The present invention encompasses single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Patent Publication Nos. WO 94/04678 and WO 94/25591; U.S. Pat. No. 6,005,079; which are incorporated herein by reference in their entireties). In one embodiment, the present invention provides single domain antibodies comprising two V_{H} domains having the amino acid sequence of any of the V_{H} domains of the EphA2 antagonistic antibodies (or an antibody that binds to EphA2 and/or decreases EphA2 cytoplasmic tail phosphorylation, and/or inhibits EphA2-ligand interaction, and/or stimulates EphA2 enzymatic activity, and/or increases cell proliferation, growth and/or survival, and/or maintains or reconstitutes cell layer integrity) with modifications such that single domain antibodies are formed. In another embodiment, the present invention also provides single domain antibodies comprising two V_{H} domains comprising one or more of the V_{H} CDRs of any of the EphA2 antagonistic antibodies or an antibody that immunospecifically binds to EphA2 and/or decreases EphA2 cytoplasmic tail phosphorylation, and/or inhibits EphA2-ligand interaction, and/or stimulates EphA2 enzymatic activity, and/or increases cell proliferation, growth and/or survival, and/or maintains or reconstitutes cell layer integrity.

Antibodies of the invention include EphA2 intrabodies (see Section 5.2.1.1). Antibody antagonistic agents of the invention that are intrabodies immunospecifically bind EphA2 and antagonize EphA2. In a more specific embodiment, an intrabody of the invention immunospecifically binds to the intracellular domain of EphA2 and decreases EphA2 cytoplasmic tail phosphorylation without causing EphA2 degradation. In another specific embodiment, the intrabody binds to the intracellular domain of EphA2 and inhibits or reduces the extent of EphA2-ligand interaction. In another specific embodiment, the intrabody binds to the intracellular domain of EphA2 and increases, accelerates and/or prolongs cell proliferation, growth and/or survival of an EphA2-expressing cell. In another specific embodiment, the intrabody binds to the intracellular domain of EphA2 and maintains/reconstitutes the integrity of an epithelial and/or endothelial cell layer.

The antibodies used in the methods of the invention may be from any animal origin including birds and mammals (e.g., human, murine, donkey, sheep, rabbit, goat, guinea pig, camel, horse, or chicken). In a most preferred embodiment, the antibody is human or has been humanized. As used herein, “human” antibodies include antibodies having the amino acid sequence of a human immunoglobulin and include antibodies isolated from human immunoglobulin libraries or from mice that express antibodies from human genes.

The antibodies used in the methods of the present invention may be monospecific, bispecific, trispecific or of greater multispecificity. Multispecific antibodies may immunospecifically bind to different epitopes of an EphA2 polypeptide or may immunospecifically bind to both an EphA2 polypeptide as well a heterologous epitope, such as a heterologous polypeptide or solid support material. See, e.g., International Patent Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

5.2.1.1 Intrabodies

In certain embodiments, the antibody to be used with the invention binds to an intracellular epitope, i.e., is an intrabody. An intrabody comprises at least a portion of an antibody that is capable of immunospecifically binding an antigen and preferably does not contain sequences coding for its secretion. Such antibodies will bind antigen intracellularly. In one embodiment, the intrabody comprises a single-chain Fv (“sFv”). sFvs are antibody fragments comprising the V_{H} and V_{L} domains of antibody, wherein these domains are present in a single polypeptide chain. Generally, the sFv polypeptide further comprises a polypeptide linker between the V_{H} and V_{L} domains which enables the sFv to form the desired structure for antigen binding. For a review of sFvs see Pluckthun in The Pharmacology ofMonoclonal Antibodies, vol. 113, Rosenburg and Moore eds. Springer-Verlag, N.Y., pp. 269-315 (1994). In a further embodiment, the intrabody preferably does not encode an operable secretory sequence and thus remains within the cell (see generally Marasco, Wash., 1998, “Intrabodies: Basic Research and Clinical Gene Therapy Applications” Springer:New York).

Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250, which references are incorporated herein by reference in their entireties. Recombinant molecular biological techniques such as those described for recombinant production of antibodies may also be used in the generation of intrabodies.

In one embodiment, intrabodies of the invention retain at least about 75% of the binding effectiveness of the complete antibody (i.e., having the entire constant domain as well as the variable regions) to the antigen. More preferably, the intrabody retains at least 85% of the binding effectiveness of the complete antibody. Still more preferably, the intrabody retains at least 90% of the binding effectiveness of the complete antibody. Even more preferably, the intrabody retains at least 95% of the binding effectiveness of the complete antibody.

In producing intrabodies, polynucleotides encoding variable region for both the V_{H} and V_{L} chains of interest can be cloned by using, for example, hybridoma mRNA or splenic mRNA as a template for PCR amplification of such domains (Huse et al., 1989, Science-246:1276). In one preferred embodiment, the polynucleotides encoding the V_{H} and V_{L} domains are joined by a polynucleotide sequence encoding a linker to make a single chain antibody (sFv). The sFv typically comprises a single peptide with the sequence V_{H}-linker-V_{L} or V_{L}-linker-V_{H}. The linker is chosen to permit the heavy chain and light chain to bind together in their proper conformational orientation (see for example, Huston, et al., 1991, Methods in Enzym. 203:46-121, which is incorporated herein by reference). In a further embodiment, the linker can span the distance between its points of fusion to each of the variable domains (e.g., 3.5 nm) to minimize distortion of the native Fv conformation. In such an embodiment, the linker is a polypeptide of at least 5 amino acid residues, at least 10 amino acid residues, at least 15 amino acid residues, or greater. In a further embodiment, the linker should not cause a steric interference with the V_{H} and V_{L} domains of the combining site. In such an embodiment, the linker is 35 amino acids or less, 30 amino acids or less, or 25 amino acids or less. Thus, in a most preferred embodiment, the linker is between 15-25 amino acid residues in length. In a further embodiment, the linker is hydrophilic and sufficiently flexible such that the V_{H} and V_{L} domains can adopt the conformation necessary to detect antigen. Intrabodies can be generated with different linker sequences inserted between identical V_{H} and V_{L} domains. A linker with the appropriate properties for a particular pair of V_{H} and V_{L} domains can be determined empirically by assessing the degree of antigen binding for each. Examples of linkers include, but are not limited to, those sequences disclosed in Table 1.

TABLE 1

| | |
|---|---|
| Sequence | SEQ ID NO. |

| | |
|---|---|
| (Gly Gly Gly Gly Ser)₃ | SEQ ID NO:1 |
| Glu Ser Gly Arg Ser Gly Gly Gly Gly Ser | SEQ ID NO:2 |
| Gly Gly Gly Gly Ser | |
| Glu Gly Lys Ser Ser Gly Ser Gly Ser Glu | SEQ ID NO:3 |
| Ser Lys Ser Thr | |
| Glu Gly Lys Ser Ser Gly Ser Gly Ser Glu | SEQ ID NO:4 |
| Ser Lys Ser Thr Gln | |
| Glu Gly Lys Ser Ser Gly Ser Gly Ser Glu | SEQ ID NO:5 |
| Ser Lys Val Asp | |
| Gly Ser Thr Ser Gly Ser Gly Lys Ser Ser | SEQ ID NO:6 |
| Glu Gly Lys Gly | |
| Lys Glu Ser Gly Ser Val Ser Ser Glu Gln | SEQ ID NO:7 |
| Leu Ala Gln Phe Arg Ser Leu Asp | |
| Glu Ser Gly Ser Val Ser Ser Glu Glu Leu | SEQ ID NO:8 |
| Ala Phe Arg Ser Leu Asp | |

In one embodiment, intrabodies are expressed in the cytoplasm. In other embodiments, the intrabodies are localized to various intracellular locations. In such embodiments, specific localization sequences can be attached to the intrabody polypeptide to direct the intrabody to a specific location. Intrabodies can be localized, for example, to the following intracellular locations: endoplasmic reticulum (Munro et al., 1987, Cell 48:899-907; Hangejorden et al., 1991, J. Biol. Chem. 266:6015); nucleus (Lanford et al., 1986, Cell 46:575; Stanton et al.,1986, PNAS 83:1772; Harlow et al., 1985, Mol. Cell Biol. 5:1605; Pap et al., 2002, Exp. Cell Res. 265:288-93); nucleolar region (Seomi et al., 1990, J. Virology 64:1803; Kubota et al., 1989, Biochem. Biophys. Res. Comm. 162:963; Siomi et al., 1998, Cell 55:197); endosomal compartment (Bakke et al., 1990, Cell 63:707-716); mitochondrial matrix (Pugsley, A. P., 1989, “Protein Targeting”, Academic Press, Inc.); Golgi apparatus (Tang et al., 1992, J. Bio. Chem. 267:10122-6); liposomes (Letourneur et al., 1992, Cell 69:1183); peroxisome (Pap et al., 2002, Exp. Cell Res. 265:288-93); trans Golgi network (Pap et al., 2002, Exp. Cell Res. 265:288-93); and plasma membrane (Marchildon et al., 1984, PNSA 81:7679-82; Henderson et al., 1987, PNSA 89:339-43; Rhee et al., 1987, J. Virol. 61:1045-53; Schultz et al., 1984, J. Virol. 133:431-7; Ootsuyama et al., 1985, Jpn. J. Can. Res. 76:1132-5; Ratner et al., 1985, Nature 313:277-84). Examples of localization signals include, but are not limited to, those sequences disclosed in Table 2.

TABLE 2

| | | |
|---|---|---|
| Localization | Sequence | SEQ ID NO. |

| | | |
|---|---|---|
| endoplasmic reticulum | Lys Asp Glu Leu | SEQ ID NO: 9 |
| endoplasmic reticulum | Asp Asp Glu Leu | SEQ ID NO: 10 |
| endoplasmic reticulum | Asp Glu Glu Leu | SEQ ID NO: 11 |
| endoplasmic reticulum | Gln Glu Asp Leu | SEQ ID NO: 12 |
| endoplasmic reticulum | Arg Asp Glu Leu | SEQ ID NO: 13 |
| nucleus | Pro Lys Lys Lys Arg Lys Val | SEQ ID NO: 14 |
| nucleus | Pro Gln Lys Lys Ile Lys Ser | SEQ ID NO: 15 |
| nucleus | Gln Pro Lys Lys Pro | SEQ ID NO: 16 |
| nucleus | Arg Lys Lys Arg | SEQ ID NO: 17 |
| nucleus | Lys Lys Lys Arg Lys | SEQ ID NO: 18 |
| nucleolar region | Arg Lys Lys Arg Arg Gln Arg Arg Arg Ala | SEQ ID NO: 19 |
| | His Gln | |
| nucleolar region | Arg Gln Ala Arg Arg Asn Arg Arg Arg Arg | SEQ ID NO: 20 |
| | Trp Arg Glu Arg Gln Arg | |
| nucleolar region | Met Pro Leu Thr Arg Arg Arg Pro Ala Ala Ser | SEQ ID NO: 21 |
| | Gln Ala Leu Ala Pro Pro Thr Pro | |
| endosomal compartment | Met Asp Asp Gln Arg Asp Leu Ile Ser Asn | SEQ ID NO: 22 |
| | Asn Glu Gln Leu Pro | |
| mitochondrial matrix | Met Leu Phe Asn Leu Arg Xaa Xaa Leu Asn | SEQ ID NO: 23 |
| | Asn Ala Ala Phe Arg His Gly His Asn Phe | |
| | Met Val Arg Asn Phe Arg Cys Gly Gln Pro | |
| | Leu Xaa | |
| peroxisome | Ala Lys Leu | SEQ ID NO: 24 |
| trans Golgi network | Ser Asp Tyr Gln Arg Leu | SEQ ID NO: 25 |
| plasma membrane | Gly Cys Val Cys Ser Ser Asn Pro | SEQ ID NO: 26 |
| plasma membrane | Gly Gln Thr Val Thr Thr Pro Leu | SEQ ID NO: 27 |
| plasma membrane | Gly Gln Glu Leu Ser Gln His Glu | SEQ ID NO: 28 |
| plasma membrane | Gly Asn Ser Pro Ser Tyr Asn Pro | SEQ ID NO: 29 |
| plasma membrane | Gly Val Ser Gly Ser Lys Gly Gln | SEQ ID NO: 30 |
| plasma membrane | Gly Gln Thr Ile Thr Thr Pro Leu | SEQ ID NO: 31 |
| plasma membrane | Gly Gln Thr Leu Thr Thr Pro Leu | SEQ ID NO: 32 |
| plasma membrane | Gly Gln Ile Phe Ser Arg Ser Ala | SEQ ID NO: 33 |
| plasma membrane | Gly Gln Ile His Gly Leu Ser Pro | SEQ ID NO: 34 |
| plasma membrane | Gly Ala Arg Ala Ser Val Leu Ser | SEQ ID NO: 35 |
| plasma membrane | Gly Cys Thr Leu Ser Ala Glu Glu | SEQ ID NO: 36 |

V_{H} and V_{L} domains are made up of the immunoglobulin domains that generally have a conserved structural disulfide bond. In embodiments where the intrabodies are expressed in a reducing environment (e.g., the cytoplasm), such a structural feature cannot exist. Mutations can be made to the intrabody polypeptide sequence to compensate for the decreased stability of the immunoglobulin structure resulting from the absence of disulfide bond formation. In one embodiment, the V_{H} and/or V_{L} domains of the intrabodies contain one or more point mutations such that their expression is stabilized in reducing environments (see Steipe et al., 1994, J. Mol. Biol. 240:188-92; Wirtz and Steipe, 1999, Protein Science 8:2245-50; Ohage and Steipe, 1999, J. Mol. Biol. 291:1119-28; Ohage et al., 1999, J. Mol Biol. 291:1129-34).

Intrabody Proteins as Therapeutics

In one embodiment, the recombinantly expressed intrabody protein is administered to a patient. Such an intrabody polypeptide must be intracellular to mediate a prophylactic or therapeutic effect. In this embodiment of the invention, the intrabody polypeptide is associated with a “membrane permeable sequence”. Membrane permeable sequences are polypeptides capable of penetrating through the cell membrane from outside of the cell to the interior of the cell. When linked to another polypeptide, membrane permeable sequences can also direct the translocation of that polypeptide across the cell membrane as well.

In one embodiment, the membrane permeable sequence is the hydrophobic region of a signal peptide (see, e.g., Hawiger, 1999, Curr. Opin. Chem. Biol. 3:89-94; Hawiger, 1997, Curr. Opin. Immunol. 9:189-94; U.S. Pat. Nos. 5,807,746 and 6,043,339, which are incorporated herein by reference in their entireties). The sequence of a membrane permeable sequence can be based on the hydrophobic region of any signal peptide. The signal peptides can be selected, e.g., from the SIGPEP database (see e.g., von Heijne, 1987, Prot. Seq. Data Anal. 1:41-2; von Heijne and Abrahmsen, 1989, FEBS Lett. 224:439-46). When a specific cell type is to be targeted for insertion of an intrabody polypeptide, the membrane permeable sequence is preferably based on a signal peptide endogenous to that cell type. In another embodiment, the membrane permeable sequence is a viral protein (e.g., Herpes Virus Protein VP22) or fragment thereof (see e.g., Phelan et al., 1998, Nat. Biotechnol. 16:440-3). A membrane permeable sequence with the appropriate properties for a particular intrabody and/or a particular target cell type can be determined empirically by assessing the ability of each membrane permeable sequence to direct the translocation of the intrabody across the cell membrane. Examples of membrane permeable sequences include, but are not limited to, those sequences disclosed in Table 3.

TABLE 3

| | |
|---|---|
| Sequence | SEQ ID NO. |

| | |
|---|---|
| Ala Ala Val Ala Leu Leu Pro Ala Val | SEQ ID NO:37 |
| Leu Leu Ala Leu Leu Ala Pro | |
| Ala Ala Val Leu Leu Pro Val Leu Leu | SEQ ID NO:38 |
| Ala Ala Pro | |
| Val Thr Val Leu Ala Leu Gly Ala Leu | SEQ ID NO:39 |
| Ala Gly Val Gly Val Gly | |

In another embodiment, the membrane permeable sequence can be a derivative. In this embodiment, the amino acid sequence of a membrane permeable sequence has been altered by the introduction of amino acid residue substitutions, deletions, additions, and/or modifications. For example, but not by way of limitation, a polypeptide may be modified, e.g., by glycosylation, acetylation, pegylation, phosphorylation, amidation, derivatization by known protecting blocking groups, proteolytic cleavage, linkage to a cellular ligand or other protein, etc. A derivative of a membrane permeable sequence polypeptide may be modified by chemical modifications using techniques known to those of skill in the art, including, but not limited to specific chemical cleavage, acetylation, formylation, metabolic synthesis of tunicamycin, etc. Further, a derivative of a membrane permeable sequence polypeptide may contain one or more non-classical amino acids. In one embodiment, a polypeptide derivative possesses a similar or identical function as an unaltered polypeptide. In another embodiment, a derivative of a membrane permeable sequence polypeptide has an altered activity when compared to an unaltered polypeptide. For example, a derivative membrane permeable sequence polypeptide can translocate through the cell membrane more efficiently or be more resistant to proteolysis.

The membrane permeable sequence can be attached to the intrabody in a number of ways. In one embodiment, the membrane permeable sequence and the intrabody are expressed as a fusion protein. In this embodiment, the nucleic acid encoding the membrane permeable sequence is attached to the nucleic acid encoding the intrabody using standard recombinant DNA techniques (see e.g., Rojas et al., 1998, Nat. Biotechnol. 16:370-5). In a further embodiment, there is a nucleic acid sequence encoding a spacer peptide placed in between the nucleic acids encoding the membrane permeable sequence and the intrabody. In another embodiment, the membrane permeable sequence polypeptide is attached to the intrabody polypeptide after each is separately expressed recombinantly (see e.g., Zhang et al., 1998, PNSA 95:9184-9). In this embodiment, the polypeptides can be linked by a peptide bond or a non-peptide bond (e.g. with a crosslinking reagent such as glutaraldehyde or a thiazolidino linkage see e.g., Hawiger, 1999, Curr. Opin. Chem. Biol. 3:89-94) by methods standard in the art.

The administration of the membrane permeable sequence-intrabody polypeptide can be by parenteral administration, e.g., by intravenous injection including regional perfusion through a blood vessel supplying the tissues(s) or organ(s) having the target cell(s), or by inhalation of an aerosol, subcutaneous or intramuscular injection, topical administration such as to skin wounds and lesions, direct transfection into, e.g., bone marrow cells prepared for transplantation and subsequent transplantation into the subject, and direct transfection into an organ that is subsequently transplanted into the subject. Further administration methods include oral administration, particularly when the complex is encapsulated, or rectal administration, particularly when the complex is in suppository form. A pharmaceutically acceptable carrier includes any material that is not biologically or otherwise undesirable, i.e., the material may be administered to an individual along with the selected complex without causing any undesirable biological effects or interacting in a deleterious manner with any of the other components of the pharmaceutical composition in which it is contained.

Conditions for the administration of the membrane permeable sequence-intrabody polypeptide can be readily be determined, given the teachings in the art (see e.g., Remington 's Pharmaceutical Sciences, 18^{th} Ed., E. W. Martin (ed.), Mack Publishing Co., Easton, Pa. (1990)). If a particular cell type in vivo is to be targeted, for example, by regional perfusion of an organ or tumor, cells from the target tissue can be biopsied and optimal dosages for import of the complex into that tissue can be determined in vitro to optimize the in vivo dosage, including concentration and time length. Alternatively, culture cells of the same cell type can also be used to optimize the dosage for the target cells in vivo.

Intrabody Gene Therapy as Therapeutic

In another embodiment, a polynucleotide encoding an intrabody is administered to a patient (e.g., as in gene therapy). In this embodiment, methods as described in Section 5.7.1 can be used to administer the polynucleotide of the invention.

5.2.1.2 Methods Of Producing Antibodies

The EphA2 antagonistic antibodies of the invention or fragments thereof can be produced by any method known in the art for the synthesis of antibodies, in particular, by chemical synthesis or, preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with EphA2 (either the full length protein or a domain thereof, e.g., the extracellular domain) and once an immune response is detected, e.g., antibodies specific for EphA2 are detected in the mouse serum, the mouse spleen is harvested and splenocytes isolated. The splenocytes are then fused by well known techniques to any suitable myeloma cells, for example cells from cell line SP20 (available from the ATCC) or NHO cells. Hybridomas are selected and cloned by limited dilution. Hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody of the invention wherein, preferably, the hybridoma is generated by fusing splenocytes isolated from a mouse immunized with EphA2 or a fragment thereof with myeloma cells and then screening the hybridomas resulting from the fusion for hybridoma clones that secrete an antibody able to bind and antagonize EphA2.

Antibody fragments which recognize specific EphA2 epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding V_{H} and V_{L} domains are amplified from animal cDNA libraries (e.g., human or murine cDNA libraries of lymphoid tissues). The DNA encoding the V_{H} and V_{L} domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the V_{H} and V_{L} domains are usually recombinantly fused to either the phage gene III or gene VIII. Phage expressing an antigen binding domain that binds to the EphA2 epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177; Kettleborough et al., 1994, Eur. J. Immunol. 24:952-958; Persic et al., 1997, Gene 187:9; Burton et al., 1994, Advances in Immunology 57:191-280; International Application No. PCT/GB91/01134; International Publication Nos. WO 90/02809, WO 91/10737, WO 92/01047, WO 92/18619, WO 93/11236, WO 95/15982, WO 95/20401, and W097/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

Phage may be screened for EphA2 binding, particularly to the extracellular domain of EphA2. Antagonistic EphA2 activities (e.g., reducing EphA2 cytoplasmic tail phosphorylation, promoting EphA2 kinase activity (other than autophosphorylation or ligand-mediated EphA2 signaling), disrupting EphA2-ligand interaction, enhancing, accelerating and/or prolonging cell proliferation, growth and/or survival, and maintaining or reconstituting epithelial and/or endothelial cell layer integrity) may also be screened (see e.g., Section 5.5 for methods of screening.)

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Patent Publication No. WO 92/22324; Mullinax et al., 1992, BioTechniques 12:864; Sawai et al., 1995, AJRI 34:26; and Better et al., 1988, Science 240:1041 (said references incorporated by reference in their entireties).

To generate whole antibodies, PCR primers including V_{H} or V_{L} nucleotide sequences, a restriction site, and a flanking sequence to protect the restriction site can be used to amplify the V_{H} or V_{L} sequences in scFv clones. Utilizing cloning techniques known to those of skill in the art, the PCR amplified V_{H} domains can be cloned into vectors expressing a V_{H} constant region, e.g., the human gamma 4 constant region, and the PCR amplified V_{L} domains can be cloned into vectors expressing a V_{L} constant region, e.g., human kappa or lambda constant regions. Preferably, the vectors for expressing the V_{H} or V_{L} domains comprise an EF-1α promoter, a secretion signal, a cloning site for the variable domain, constant domains, and a selection marker such as neomycin. The V_{H} and V_{L} domains may also be cloned into one vector expressing the necessary constant regions. The heavy chain conversion vectors and light chain conversion vectors are then co-transfected into cell lines to generate stable or transient cell lines that express fill-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Patent Publication Nos. WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Patent Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Medarex (Princeton, N.J.) can be engaged to provide human antibodies directed against a selected antigen using technology similar to that described above.

A chimeric antibody is a molecule in which different portions of the antibody are derived from different immunoglobulin molecules such as antibodies having a variable region derived from a non-human antibody and a human immunoglobulin constant region. Methods for producing chimeric antibodies are known in the art. See e.g., Morrison, 1985, Science 229:1202; Oi et al., 1986, Bio Techniques 4:214; Gillies et al., 1989, J. Immunol. Methods 125:191-202; and U.S. Pat. Nos. 5,807,715, 4,816,567, and 4,816,397, which are incorporated herein by reference in their entirety. Chimeric antibodies comprising one or more CDRs from a non-human species and framework regions from a human immunoglobulin molecule can be produced using a variety of techniques known in the art including, for example, CDR-grafting (EP 239,400; International Patent Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, PNSA 91:969), and chain shuffling (U.S. Pat. No. 5,565,332). In one embodiment, a chimeric antibody of the invention immunospecifically binds EphA2 and comprises one, two, or three V_{L} CDRs having an amino acid sequence of any of the V_{L} CDRs of an antibody of the invention within human framework regions. In another embodiment, a chimeric antibody of the invention immunospecifically binds EphA2 and comprises one, two, or three V_{H} CDRs having an amino acid sequence of any of the V_{H} CDRs of an antibody of the invention within human framework regions. In another embodiment, a chimeric antibody of the invention immunospecifically binds EphA2 and comprises one, two, or three V_{L} CDRs having an amino acid sequence of any of the V_{L} CDRs of an antibody of the invention and further comprises one, two, or three V_{H} CDRs having an amino acid sequence of any of the V_{H} CDRs of an antibody of the invention within human framework regions. In a preferred embodiment, a chimeric antibody of the invention immunospecifically binds EphA2 and comprises three V_{L} CDRs having an amino acid sequence of any of the V_{L} CDRs of an antibody of the invention and three V_{H} CDRs having an amino acid sequence of any of the V_{H} CDRs of an antibody of the invention within human framework regions.

Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann et al., 1988, Nature 332:323, which are incorporated herein by reference in their entireties.)

5.2.2 EphA2 Fragments As Polypeptide Antagonistic Agents

In another embodiment, the EphA2 antagonistic agent is a fragment of the EphA2 polypeptide. Because EphA2 bound to its endogenous ligand, Ephrin A1, causes a decrease in cell growth or proliferation, any method that decreases the amount of EphA2-Ephrin A1 binding is encompassed in the methods of the invention. In one embodiment, a fragment of EphA2 which retains its ability to bind Ephrin A1 is used in the methods of the invention to inhibit binding of cellular EphA2 from binding to cellular Ephrin A1 (e.g., the EphA2 extracellular domain). In another embodiment, a fusion protein comprises the fragment of EphA2 which retains its ability to bind Ephrin A1 (e.g., the extracellular domain of EphA2 fused to immunoglobulin heavy chain, see Carles-Kinch et al., 2002, Cancer Res. 62:2840-7). In a preferred embodiment, the EphA2 fragment is soluble. Fragments of EphA2 can be made (e.g., using EphA2 sequences known in the art such as Genbank Accession No. BC037166) and assayed for the ability to bind Ephrin A1. In one embodiment, the fragment comprises amino acid residues 1 to approximately 400, 500, or 600. In a more specific embodiment, the fragment is amino acid residues 1-534 of EphA2. Any method known in the art to detect binging between proteins may be used including, but not limited to, affinity chromatography, size exclusion chromatography, electrophoretic mobility shift assay. Polypeptide antagonistic agents of the invention that are EphA2 fragments include polypeptides that are 100%, 98%, 95%, 90%, 85%, 80%, 75%, 70%, 65%, 60%, 55%, 50%, 45%, 40% identical to endogenous EphA2 sequences. The determination of percent identity of two amino acid sequences can be determined by any method known to one skilled in the art, including BLAST protein searches.

5.2.3 Modified Polypeptide Antagonistic Agents

The polypeptide antagonistic agents used in the methods of the invention (e.g., antibodies or EphA2 fragments) include derivatives that are modified, i.e, by the covalent attachment of any type of molecule to the polypeptide. For example, but not by way of limitation, the polypeptide derivatives include polypeptides that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The methods of the present invention also encompass the use of polypeptide EphA2 antagonistic agents that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the polypeptide antagonistic agents in mammals, preferably humans, results in a higher concentration of said polypeptide antagonistic agents in the mammals, and thus, reduces the frequency of the administration of said polypeptide antagonistic agents and/or reduces the amount of said polypeptide antagonistic agents to be administered. Polypeptide antagonistic agents having increased in vivo half-lives can be generated by techniques known to those of skill in the art. For example, polypeptide antagonistic agents with increased in vivo half-lives can be generated by modifying (e.g., substituting, deleting or adding) amino acid residues. In one embodiment, when the polypeptide antagonistic agent is an antibody, such amino acid residues to be modified can be those residues involved in the interaction between the Fc domain and the FcRn receptor (see, e.g., International Patent Publication No. WO 97/34631 and U.S. patent application Ser. No. 10/020,354 filed Dec. 12, 2001 entitled “Molecules With Extended Half-Lives, Compositions and Uses Thereof,” which are incorporated herein by reference in their entireties). Polypeptide antagonistic agents with increased in vivo half-lives can also be generated by attaching to said polypeptides polymer molecules such as high molecular weight polyethylene glycol (PEG). PEG can be attached to said polypeptide antagonistic agents with or without a multifunctional linker either through site-specific conjugation of the PEG to the—or C-terminus of said polypeptide or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the polypeptide antagonistic agents. Unreacted PEG can be separated from polypeptide antagonistic agent-PEG conjugates by, e.g., size exclusion or ion-exchange chromatography.

5.2.3.1 Polynucleotides Encoding Polypeptide Antagonistic Agents

The EphA2 polypeptide antagonistic agents of the invention include polypeptides produced from polynucleotides that hybridize to polynucleotides which encode polypeptides disclosed in sections 5.2.1 and 5.2.2 above. In one embodiment, antibodies of the invention include EphA2 monoclonal antibodies produced from polynucleotides that hybridize to polynucleotides encoding monoclonal antibodies that antagonize EphA2 in one or more of the assays described in Section 5.5. In another embodiment, EphA2 fragments used in the methods of the invention include polypeptides produced from polynucleotides that hybridize to polynucleotides encoding a ligand binding domain of EphA2. Conditions for hybridization include, but are not limited to, stringent hybridization conditions such as hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The polynucleotides encoding antibodies of the invention or the EphA2 fragments used in the methods of the invention may be obtained and sequenced by any method known in the art. Such a polynucleotide encoding a polypeptide antagonistic agent used in the methods of the invention may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the polypeptide, annealing and ligating of those oligonucleotides, and then amplification of the ligated oligonucleotides by PCR.

Alternatively, a polynucleotide encoding polypeptide antagonistic agent used in the methods of the invention may be generated from nucleic acid from a suitable source. If a clone containing a nucleic acid encoding a particular polypeptide is not available, but the sequence of the polypeptide is known, a nucleic acid encoding the polypeptide may be chemically synthesized or obtained from a suitable source (e.g., an antibody cDNA library, or a cDNA library generated from, or nucleic acid, preferably poly A+RNA, isolated from, any tissue or cells expressing the desired polypeptide, such as hybridoma cells selected to express an antibody of the invention or epithelial and/or endothelial cells that express EphA2) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or EphA2 polypeptide. Amplified nucleic acids generated by PCR may then be cloned into replicable cloning vectors using any method well known in the art.

Once the nucleotide sequence of the polypeptide antagonistic agent used in the methods of the invention is determined, the nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate polypeptides having a different amino acid sequence, for example to create amino acid substitutions, deletions, and/or insertions.

Standard techniques known to those skilled in the art can be used to introduce mutations in the nucleotide sequence encoding a polypeptide antagonistic agent including, e.g., site-directed mutagenesis and PCR-mediated mutagenesis, which results in amino acid substitutions. Preferably, the derivatives include less than 15 amino acid substitutions, less than 10 amino acid substitutions, less than 5 amino acid substitutions, less than 4 amino acid substitutions, less than 3 amino acid substitutions, or less than 2 amino acid substitutions relative to the original antibody or fragment thereof. In a preferred embodiment, the derivatives have conservative amino acid substitutions made at one or more predicted non-essential amino acid residues.

The present invention also encompasses the use of antibodies or antibody fragments comprising the amino acid sequence of any EphA2 antagonistic antibodies described above with mutations (e.g., one or more amino acid substitutions) in the framework or variable regions. Preferably, mutations in these antibodies maintain or enhance the avidity and/or affinity of the antibodies for the particular antigen(s) to which they immunospecifically bind. Standard techniques known to those skilled in the art (e.g., immunoassays or ELISA assays) can be used to assay the degree of binding between a polypeptide antagonistic agent and its binding partner. In a specific embodiment, when a polypeptide antagonistic agent is an antibody, binding to an EphA2 antigen can be assessed. In another embodiment, when a polypeptide antagonistic agent is an EphA2 fragment, binding to Ephrin A1 can be assessed.

5.2.3.2 Recombinant Production of Polypeptide Antagonistic Agents

Recombinant expression of a polypeptide antagonistic agent (including, but not limited to derivatives, analogs or fragments thereof) requires construction of an expression vector containing a polynucleotide that encodes the polypeptide. Once a polynucleotide encoding a polypeptide antagonistic agent has been obtained, a vector for the production of the polypeptide antagonistic agent may be produced by recombinant DNA technology using techniques well known in the art. Methods which are well known to those skilled in the art can be used to construct expression vectors containing polypeptide coding sequences and appropriate transcriptional and translational control signals. Thus, methods for preparing a protein by expressing a polynucleotide containing are described herein. These methods include, for example, in vitro recombinant DNA techniques, synthetic techniques, and in vivo genetic recombination. The invention, thus, provides replicable vectors comprising a nucleotide sequence encoding a EphA2 antagonistic polypeptide agent.

The expression vector is transferred to a host cell by conventional techniques and the transfected cells are then cultured by conventional techniques to produce a polypeptide antagonistic agent. Thus, the invention includes host cells containing a polynucleotide encoding a polypeptide antagonistic agent or fragments thereof operably linked to a heterologous promoter.

A variety of host-expression vector systems may be utilized to express polypeptide antagonistic agents (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express a polypeptide antagonistic agent of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing polypeptide antagonistic agent coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant polypeptide antagonistic agent, are used for the expression of a polypeptide antagonistic agent. For example, mammalian cells such as Chinese hamster ovary cells (CHO), in conjunction with a vector such as the major intermediate early gene promoter element from human cytomegalovirus is an effective expression system for polypeptide antagonistic agents, especially antibody polypeptide antagonistic agents (Foecking et al., 1986, Gene 45:101; and Cockett et al., 1990, BioTechnology 8:2). In a specific embodiment, the expression of nucleotide sequences encoding a polypeptide antagonistic agent is regulated by a constitutive promoter, inducible promoter or tissue specific promoter.

In bacterial systems, a number of expression vectors may be advantageously selected depending upon the use intended for the polypeptide being expressed. For example, when a large quantity of such a protein is to be produced, for the generation of pharmaceutical compositions, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the polypeptide coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the polypeptide antagonistic agent in infected hosts (e.g., see Logan & Shenk, 1984, PNSA 8 1:355-359). Specific initiation signals may also be required for efficient translation of inserted polypeptide coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the polypeptide antagonistic agent. Such engineered cell lines may be particularly useful in screening and evaluation of compositions that interact directly or indirectly with the polypeptide antagonistic agent.

A number of selection systems may be used, including but not limited to, the herpes simplex virus thymidine kinase (Wigler et al., 1977, Cell 11:223), glutamine synthetase, hypoxanthine guanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, gs-, hgprt- or aprt- cells, respectively. Also, antimetabolite resistance can be used as the basis of selection for the following genes: dhfr, which confers resistance to methotrexate (Wigler et al., 1980, PNSA 77:357; O'Hare et al., 1981, PNSA 78:1527); gpt, which confers resistance to mycophenolic acid (Mulligan & Berg, 1981, PNSA 78:2072); neo, which confers resistance to the aminoglycoside G-418 (Wu and Wu, 1991, Biotherapy 3:87; Tolstoshev, 1993, Ann. Rev. Pharmacol. Toxicol. 32:573; Mulligan, 1993, Science 260:926; and Morgan and Anderson, 1993, Ann. Rev. Biochem. 62: 191; May, 1993, TIB TECH 11:155-); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of a polypeptide antagonistic agent can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing polypeptide antagonistic agent is amplifiable, increase in the level of inhibitor present in culture of host cell will increase the number of copies of the marker gene. Since the amplified region is associated with the polypeptide antagonistic agent gene, production of the polypeptide antagonistic agent will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, PNSA 77:2197). The coding sequences for the heavy and light chains may comprise cDNA or genomic DNA.

Once a polypeptide antagonistic agent of the invention has been produced by recombinant expression, it may be purified by any method known in the art for purification of a polypeptide, for example, by chromatography (e.g., ion exchange, affinity, and sizing column chromatography), centrifugation, differential solubility, or by any other standard technique for the purification of proteins. Further, the polypeptide antagonistic agents may be fused to heterologous polypeptide sequences described herein or otherwise known in the art to facilitate purification.

Polypeptide antagonistic agents of the invention that are antibodies may be expressed using vectors which already include the nucleotide sequence encoding the constant region of the antibody molecule (see, e.g., U.S. Pat. Nos. 5,919,900; 5,747,296; 5,789,178; 5,591,639; 5,658,759; 5,849,522; 5,122,464; 5,770,359; 5,827,739; International Patent Publication Nos. WO 89/01036; WO 89/10404; Bebbington et al., 1992, BioTechnology 10:169). The variable domain of the antibody may be cloned into such a vector for expression of the entire heavy, the entire light chain, or both the entire heavy and light chains. In preferred embodiments for the expression of double-chained antibodies, vectors encoding both the heavy and light chains may be co-expressed in the host cell for expression of the entire immunoglobulin molecule.

5.3 Polynucleotide Antagonistic Agents

In addition to EphA2 polypeptide antagonistic agents of the invention, nucleic acid molecules can be used in methods of the invention. Because EphA2 bound to its endogenous ligand, Ephrin A1, causes a decrease in cell growth or proliferation, any method that 1) decreases or disrupts EphA2-Ephrin A1 binding; 2) upregulates EphA2 expression such that the amount of EphA2 on the cell surface exceeds the amount of natural ligand available for binding and thus increases the amount of unbound EphA2; or 3) decreases Ephrin A1 expression such that amount of natural ligand available to bind EphA2 is decreased and thus increases the amount of unbound EphA2 is encompassed in the methods of the invention. In one embodiment, the amount of endogenous ligand available for binding to EphA2 is decreased. Any method known in the art to decrease expression of EphA2 ligand, Ephrin A1, can be used in the methods of the invention including, but not limited to, antisense and RNA interference technology. Thus, EphA2 antagonistic agents encompasses those agents that serve to decrease Ephrin A1 expression or availability for EphA2-binding.

5.3.1 Antisense

The present invention encompasses Ephrin A1 antisense nucleic acid molecules, i.e., molecules which are complementary to all or part of a sense nucleic acid encoding Ephrin A1, molecules which are complementary to the coding strand of a double-stranded Ephrin A1 cDNA molecule or molecules complementary to an Ephrin A1 MRNA sequence (e.g., human Ephrin A1 mRNA sequence at Genbank Accession No. BC032698). Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

02] An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid can be produced biologically using an expression vector into which a nucleic acid has been subdloned in an antisense orientation (i.e., RNA transcribed from the inserted nucleic acid will be of an antisense orientation to a target nucleic acid of interest, i.e., Ephrin A1).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an aL-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids Res. 15:6625). The antisense nucleic acid molecule can also comprise a 2′-o-methylribonucleotide (Inoue et al., 1987, Nucleic Acids Res. 15:6131) or a chimeric RNA-DNA analogue (Inoue et al., 1987, FEBS Lett. 215:327).

5.3.2 RNA Interference

In certain embodiments, an RNA interference (RNAi) molecule is used to decrease Ephrin A1 expression. RNAi is defined as the ability of double-stranded RNA (dsRNA) to suppress the expression of a gene corresponding to its own sequence. RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA (e.g., human Ephrin A1 mRNA sequence at Genbank Accession No. BC032698). This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

06] Double-stranded (ds) RNA can be used to interfere with gene expression in mammals (Wianny & Zemicka-Goetz, 2000, Nature Cell Biology 2: 70-75; incorporated herein by reference in its entirety). dsRNA is used as inhibitory RNA or RNAi of the function of Ephrin A1 to produce a phenotype that is the same as that of a null mutant of Ephrin A1 (Wianny & Zemicka-Goetz, 2000, Nature Cell Biology 2: 70-75). In certain embodiments, dsDNA encoding dsRNA (e.g., as hairpin structures) is used to express RNAi-mediating dsDNA in the cell.

5.4 Prophylactic/Therapeutic Methods

The present invention encompasses methods for treating, managing, or preventing a hypoproliferative cell disorder or disorder involving increased cell death, especially those disorders relating to EphA2 underexpreesion or the destruction, shedding, or inadequate proliferation/growth of epithelial and/or endothelial cells (particularly IC and lesions associated with IBD), in a subject comprising administering one or more EphA2 antagonistic agent or EphA2 cell proliferation stimulative agents of the invention. In one embodiment, one or more EphA2 agents can be administered in combination with one or more other therapeutic agents useful in the treatment, management, or prevention of a hypoproliferative cell disorder or disorder involving increased cell death.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity and type of hypoproliferative cell disorder or disorder involving increased cell death relating to the destruction and/or shedding of epithelial and/or endothelial cells, the route of administration, as well as age, body weight, response, and the past medical history of the patient. Suitable regimens can be selected by one skilled in the art by considering such factors and by following, for example, dosages reported in the literature and recommended in the Physician's Desk Reference (56^{th} ed., 2002).

5.4.1 Patient Population

The present invention encompasses methods for treating, managing, or preventing a hypoproliferative cell disorder or disorder involving increased cell death, especially those disorders relating to the destruction, shedding, or inadequate proliferation of epithelial and/or endothelial cells (particularly IC and lesions associated with IBD), in a subject comprising administering one or more EphA2 antagonistic agent or EphA2 cell proliferation stimulative agents of the invention. The subject is preferably a mammal such as non-primate (e.g., cows, pigs, horses, cats, dogs, rats, etc.) and a primate (e.g., monkey, such as a cynomolgous monkey and a human). In a preferred embodiment, the subject is a human.

The methods and compositions of the invention comprise the administration of one or more EphA2 antagonistic agents of the invention to patients suffering from or expected to suffer from (e.g., have a genetic predisposition for or previously suffered from) a hypoproliferative cell disorder or disorder involving increased cell death, especially those disorders relating to the destruction, shedding, or inadequate proliferation of epithelial and/or endothelial cells (particularly IC and lesions associated with IBD). Such patients may have been previously treated or are currently being treated for the hypoproliferative cell disorder or disorder involving increased cell death, e.g., with a non-EphA2-based therapeutic. The methods and compositions of the invention may be used as a first line or second line therapeutic. The methods and compositions of the invention can be used before any adverse effects or intolerance of the non-EphA2-based therapies occurs. The invention also encompasses methods for administering one or more EphA2 agents of the invention to prevent the onset or recurrence of a hypoproliferative cell disorder or disorder involving increased cell death in patients predisposed to having a hypoproliferative epithelial and/or endothelial cell disorder.

In one embodiment, the patient has been or is currently being treated for IC with a non-EphA2-based therapeutic, e.g., analgesics such as pentzocine (Talwin™), propiram ftimarate (Dirame™), tramadol (Ultram™), gabapentin (Neurontin™), mexiletine (Mextie™), prochlorperazine (Compazine™), phenazopyridine hydrochloride (Pyridium™); antidepressants such as amitriptyline (Elavil™), desipramine (Norpramin™), doxepin (Sinequan™), imipramine (Tofranil™); antispasmodics such as hyoscyamine sulfate (Anaspaz™), oxybutynin chloride (Ditropan™), flavoxate hydrochloride (Urispas™), atropine sulfate (Urised™); antihormones such as leuprolide acetate (Lupron™), tamoxifen (Nolvadex™); anti-inflammatory agents such as celecoxib (Celebrex™), choline magnesium trisalicylate (Trilisate™), chrondrotin sulphate+quercetin (Algonot-Plus™), dipyrone (Novalgin™), rofecoxib (Vioxx™); leukotriene blockers such as montelukast (Singulair™), zafirlukast (Accolate™), zileuton (Zyflo™); immunosuppressive agents such as cyclosporin (Neoral™), etanercept (Embrel™), inflixmab (Remicade™), methotrexate; mast cell mediator release/action inhibitors such as cimetide (Tagamet™), cromolyn (Intal™, Gastrocrom™), hydroxzine (Atarax™, Vistaril™), indolinone derivaties (SUGEN™), IPD-1151T; mucosal surface protectors such as heparin, hyaluronic acid (Cystistat™), pentosanpolysulphate (Elmiron™), prostagandin E₁ analogues (Misoprostol™); neuropeptide depletors/receptor antagonists such as resiniferatoxin, neurokinin receptor antagonists (CP 96,345, SR-48,968), neurotensin receptor antagonists (SR-48,692); neuroplytic/antineuronal such as antalarmin, astessin, histamine-3 receptor agonists (BP 2-94), tizanidine (Zanaflex™); L-Arginine; Bacillus Calmette-Guerin; Doxorubicin (Adriamycin™); octreotide (Sandostatin™); and/or DMSO (Rimso-50™) (see Theoharides and Sant, 2001, Exp. Opin. Invest. Drugs 10:521-46).

In another embodiment, the patient has been or is currently being treated for IBD with a non-EphA2-based therapeutic, e.g., aminosalisylate such as sulfasalazine; mesalamine such as pentasa, asacol, rowasa, olsalazine, balsalazide; steroids and steroid analogues such as budesonide; immunosuppressive agents such as azathiporine, 6-mercaptopurine, cyclosporine, methotrexate; fish oils such as eicosapentaenoic acid; antimicrobials such as ciprofloxin, metronidazole; TNFα inhibitors such as inflixmab; IL-2 inhibitors; heparin ; olsalazine; and/or nicotine (see Wolf and Lashner, 2002, Cleveland Clinic Journal ofMedicine 69:621-31).

The present invention also encompasses methods for administering one or more EphA2 agents of the invention to treat or ameliorate symptoms of a hypoproliferative cell disorder or disorder involving increased cell death in patients that are or have become refractory to non-EphA2-based therapies. The determination of whether the symptoms are refractory can be made either in vivo or in vitro by any method known in the art for assaying the effectiveness of treatment on affected cells in the hypoproliferative cell disorder or disorder involving increased cell death, particularly epithelial and/or endothelial cells.

5.4.2 Other Prophylactic/Therapeutic Agents

In certain embodiments, the invention provides methods for treating a patient's hypoproliferative cell disorder or disorder involving increased cell death (e.g., IC or lesions associated with IBD) by administering one or more EphA2 agents of the invention in combination with any other therapy that reduces the symptoms of a hypoproliferative cell disorder or disorder involving increased cell death. Administration of the therapeutic/prophylactic agents to a patient can be at exactly the same time or in a sequence within a time interval such that the agents can act together to provide an increased benefit than if they were administered otherwise. For example, each therapeutic/prophylactic agent may be administered in any order at different points in time; however, if not administered at the same time, they should be administered sufficiently close in time so as to provide the desired therapeutic or prophylactic effect. Each therapeutic/prophylactic agent can be administered separately, in any appropriate form and by any suitable route.

In various embodiments, the prophylactic or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same patient visit.

In one embodiment, EphA2 antagonistic agents of the invention are administered in combination with a therapy currently known to treat a hypoproliferative cell disorder or disorder involving increased cell death (see e.g., Section 5.4.1 supra). In another embodiment, EphA2 antagonistic agents of the invention are administered in combination with an immunomodulatory agent or an anti-urinary tract infection agent. In another embodiment, EphA2 antagonistic agents of the invention are administered in combination with a therapy currently known to treat a hypoproliferative cell disorder or disorder involving increased cell death and an immunomodulatory or an anti-urinary tract infection agent.

5.4.2.1 Immunomodulatory Agents

In certain embodiments, the present invention provides compositions comprising one or more EphA2 agents of the invention and one or more immunomodulatory agents (i.e., agents which modulate the immune response in a subject), and methods for treating disorders involving hypoproliferative cells in a subject comprising the administration of said compositions or administration of an EphA2-based prophylactic/therapeutic in combination with one or more immunomodulatory agents. In a specific embodiment of the invention, the immunomodulatory agent inhibits or suppresses the immune response in a human subject. Immunomodulatory agents are well-known to one skilled in the art and can be used in the methods and compositions of the invention.

Immunomodulatory agents can affect one or more or all aspects of the immune response in a subject. Aspects of the immune response include, but are not limited to, the inflammatory response, the complement cascade, leukocyte and lymphocyte proliferation, monocyte and/or basophil counts, and cellular communication among cells of the immune system. In certain embodiments of the invention, an immunomodulatory agent modulates one aspect of the immune response. In other embodiments, an immunomodulatory agent modulates more than one aspect of the immune response. In a preferred embodiment of the invention, the administration of an immunomodulatory agent to a subject inhibits or reduces one or more aspects of the subject's immune response capabilities.

In accordance with the invention, one or more immunomodulatory agents can be administered to a subject with a hypoproliferative cell disorder or disorder involving increased cell death prior to, subsequent to, or concomitantly with an EphA2 antagonistic agent of the invention. Preferably, one or more immunomodulatory agents are administered to a subject with a hypoproliferative cell disorder or disorder involving increased cell death to reduce or inhibit one or more aspects of the immune response as necessary. Any technique well-known to one skilled in the art can be used to measure one or more aspects of the immune response, and thereby determine when it is necessary to administer an immunomodulatory agent. In a preferred embodiment, one or more immunomodulatory agents are administered to a subject with a hypoproliferative epithelial and/or endothelial cell disorder so as to transiently reduce or inhibit one or more aspects of the immune response. Such a transient inhibition or reduction of one or more aspects of the immune system can last for hours, days, weeks, or months. The transient reduction or inhibition of one or more aspects of the immune response potentiates the therapeutic effect of the EphA2 antagonistic agent of the invention.

In a preferred embodiment, the immunomodulatory agent decreases the amount of IL-9. In a more preferred embodiment, the immunomodulatory agent is an antibody (preferably a monoclonal antibody) or fragment thereof that immunospecifically binds to IL-9 (see e.g., U.S. patent application Ser. No. ______ filed Apr. 12, 2004 entitled “Methods of Preventing or Treating Respiratory Conditions” by Reed (Attorney Docket No. 10271-113-999), U.S. patent application Ser. No. ______ filed Apr. 12, 2004 entitled “Recombinant IL-9 Antibodies and Uses Thereof” by Reed (Attorney Docket No. 10271-112-999), and U.S. patent application Ser. No. ______ filed Apr. 12, 2004 entitled “Anti-IL-9 Antibody Formulations and Uses Thereof” by Reed (Attorney Docket No. 10271-126-999), all of which are incorporated by reference herein in their entireties. Although not intending to be bound by a particular mechanism of action, the use of anti-IL-9 antibodies neutralize the ability of IL-9 to have a biological effect and thereby blocks or decreases inflammatory cell recruitment.

In other embodiments, other immunomodulatory agents which can be used in the compositions and methods of the invention can be those that are commercially available and known to function as immunomodulatory agents. The immunomodulatory agents include, but are not limited to, agents such as cytokines, antibodies (e.g., human, humanized, chimeric, monoclonal, polyclonal, Fvs, sFvs, Fab or F(ab)2 fragments or epitope binding fragments), inorganic compounds, or peptide mimetics. Further exanples of immunomodulatory agents include, but are not limited to, anti-IL-13 monoclonal antibodies, anti-IL-4 monoclonal antibodies, anti-IL-5 monoclonal antibodies, anti-IL-2R antibodies (e.g., anti-Tac monoclonal antibody and BT 536), anti-CD4 monoclonal antibodies, anti-CD3 monoclonal antibodies, the anti-CD3 monoclonal human antibody OKT3, anti-CD8 monoclonal antibodies, anti-CD40 ligand monoclonal antibodies, anti-CD2 monoclonal antibodies (e.g., International Patent Publication WO 02/070007 published Sep. 12, 2002), CTLA4-immunoglobulin, cyclophosphamide, cyclosporine A, macrolide antibiotics (e.g., FK506 (tacrolimus)), methylprednisolone (MP), corticosteroids, mycophenolate mofetil, rapamycin (sirolimus), mizoribine, deoxyspergualin, brequinar, malononitriloamindes.(e.g., leflunamide), beta 2-agonists, leukotriene antagonists, and agents that decrease IgE levels.

The immunomodulator activity of an immunomodulatory agent can be determined in vitro and/or in vivo by any technique well-known to one skilled in the art, including, e.g., by CTL assays, proliferation assays, immunoassays (e.g. ELISAs) for the expression of particular proteins such as co-stimulatory molecules and cytokines, and FACS.

5.4.2.2 Anti-Urinary Tract Infection Agents

In certain embodiments, the present invention provides compositions comprising one or more EphA2 antagonistic agents of the invention and one or more anti-urinary tract infection (UTI) agents (i.e., agents which decrease or inhibit the occurrence or recurrence of UTIs in a subject), and methods for treating disorders involving hypoproliferative cells in a subject comprising the administration of said compositions or administration of an EphA2-based prophylactic/therapeutic in combination with one or more anti-UTI agents.

E. coli colonization of the urinary epithelium is a required step in the acquisition and progression of E. coli UTIs. In a typical course of E. coli urinary tract infection, bacteria originate from the bowel, ascend into the bladder, and adhere to the bladder mucosa where they multiply and establish an infection before ascending into the ureters and kidneys. The initiation and persistence of many bacterial infections depends on a stereo-chemical fit between an adhesin located at the bacteria's pilus tip and specific receptor architectures on host cells. Uropathogenic strains of E. coli express P and type 1 pili that bind to receptors present in uroepithelial cells. The adhesin present at the tip of the P pilus, PapG, binds to the Galα(1-4)Gal moiety present in the globoseries of glycolipids. Alternatively, the type 1 adhesin, FimH, binds D-mannose present in glycolipids and glycoproteins.

Anti-UTI agents are well-known to one skilled in the art and can be used in the methods and compositions of the invention. For example, disruption or prevention of pilus-mediated attachment of E. coli to urinary epithelia prevents or retard the development of UTI. In certain embodiments, antibodies directed against FimH (for type 1 pili) or PapG (for P pili) can be directly administered or induced via immunization to prevent or treat UTIs (see e.g., International Patent Publication Nos. WO 01/05978 entitled “FimH Adhesin-based Vaccines”; WO 01/04148 entitled “Donor Strand Complemented Pilin and Adhesin Broad-based Vaccines”; WO 02/15928 entitled “Method of Administering FimH Protein as a Vaccine for Urinary Tract Infections”; WO 02/04496 entitled “FimH Adhesin Proteins and Methods of Use”; U.S. Pat. Nos. 10/015,166 and 10/015,085, filed Dec. 10, 2001 All of which are incorporated by reference herein in their entireties).

5.4.3 Coniugated Antibodies

The present invention encompasses the use of an antibody to target a prophylactic/therapeutic agent to cells involved in the hypoproliferative disorder to be treated (e.g., hypoproliferating epithelial and/or endothelial cells). Such therapeutic agents are recombinantly fuised or chemically conjugated (including both covalent and non-covalent conjugations) to an antibody or a fragment thereof (e.g., Fab fragment, Fd fragment, Fv fragment, F(ab)₂ fragment, or portion thereof). In one embodiment, an EphA2 antagonistic antibody of the invention or fragment thereof is conjugated to a prophylactic/therapeutic agent used to treat the hypoproliferative disorder. Such prophylactic/therapeutic agents can be EphA2-based (e.g., antagonistic agents of the invention) or non-EphA2-based (e.g., non-EphA2-based agents currently administered to treat the disorder, analgesic agents, anesthetic agents, antibiotics, immunomodulatory agents or anti-urinary tract infection agents). In another embodiment, an antibody or fragment thereof that targets to the epithelial and/or endothelial cells affected by the hypoproliferative disorder (e.g., through recognition of a pathology-associated marker) but does not immunospecifically bind EphA2 is conjugated to a prophylactic/therapeutic agent used to treat the hypoproliferative disorder. Such prophylactic/therapeutic agents are EphA2-based (e.g., antagonistic agents of the invention).

A conjugated agent's relative efficacy in comparison to the free agent can depend on a number of factors. For example, rate of uptake of the antibody-agent into the cell (e.g., by endocytosis), rate/efficiency of release of the agent from the antibody, rate of export of the agent from the cell, etc. can all effect the action of the agent. Antibodies used for targeted delivery of agents can be assayed for the ability to be endocytosed by the relevant cell type (i.e., the cell type associated with the disorder to be treated) by any method known in the art. Additionally, the type of linkage used to conjugate an agent to an antibody should be assayed by any method known in the art such that the agent action within the target cell is not impeded.

In another embodiment, antibodies can be fused or conjugated to liposomes, wherein the liposomes are used to encapsulate therapeutic agents (see e.g., Park et al., 1997, Can. Lett. 118:153-160; Lopes de Menezes et al., 1998, Can. Res. 58:3320-30; Tseng et al., 1999, Int. J. Can. 80:723-30; Crosasso et al., 1997, J. Pharm. Sci. 86:832-9). In a preferred embodiment, the pharmokinetics and clearance of liposomes are improved by incorporating lipid derivatives of PEG into liposome formulations (see e.g., Allen et al., 1991, Biochem Biophys Acta 1068:133-41; Huwyler et al., 1997, J. Pharmacol. Exp. Ther. 282:1541-6).

Therapeutic agents can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv. Drug Deliv. Rev. 53:171-216). Additional techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58. Methods for fusing or conjugating antibodies to polypeptide agents are known in the art. See, e.g., U.S. Pat. Nos. 5,336,603, 5,622,929, 5,359,046, 5,349,053, 5,447,851, and 5,112,946; EP 307,434; EP 367,166; International Patent Publication Nos. WO 96/04388 and WO 91/06570; Ashkenazi et al., 1991, PNSA 88: 10535-10539; Zheng et al., 1995, J. Immunol. 154:5590-5600; and Vil et al., 1992, PNSA 89:11337-11341. Methods for fusing or conjugating antibodies to conjugated to another antibody are described by Segal in U.S. Pat. No. 4,676,980. The fusion of an antibody to a agent does not necessarily need to be direct, but may occur through linker sequences. Such linker molecules are commonly known in the art and described in Denardo et al., 1998, Clin Cancer Res. 4:2483-90; Peterson et al., 1999, Bioconjug. Chem. 10:553; Zimmerman et al., 1999, Nucl. Med. Biol. 26:943-50; Garnett, 2002, Adv. Drug Deliv. Rev. 53:171-216.

In other embodiments, antibody properties can be altered as desired (e.g., antibodies or fragments thereof with higher affinities and lower dissociation rates) through the techniques of gene-shuffling, motif-shuffling, exon-shuffling, and/or codon-shuffling (collectively referred to as “DNA shuffling”). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308. Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions immunospecifically bind to an antigen expressed on a cell associated with a particular disorder may be recombined with one or more components, motifs, sections, parts, domains, fragments, etc. of one or more heterologous molecules.

In other embodiments, the conjugated antibodies or fragments thereof can be additionally fused to marker sequences, such as a peptide, to facilitate purification. In preferred embodiments, the marker amino acid sequence is a hexa-histidine peptide, such as the tag provided in a pQE vector (QIAGEN, Inc., Chatsworth, Calif.), among others, many of which are commercially available (see e.g., Gentz et al., 1989, PNSA 86:821). Other peptide tags useful for purification include, but are not limited to, the hemagglutinin (HA) tag, which corresponds to an epitope derived from the influenza hemagglutinin protein (Wilson et al., 1984, Cell 37:767) and the “flag” tag. Any purification method known in the art can be used (see e.g., International Patent Publication WO 93/21232; EP 439,095; Naramura et al., 1994, Immunol. Lett. 39:91-99; U.S. Pat. No. 5,474,981; Gillies et al., 1992, PNSA 89:1428-1432; and Fell et al., 1991, J. Immunol. 146:2446-2452).

In other embodiments, conjugated antibodies or fragments or variants thereof can be conjugated to a diagnostic or detectable agent either alone or in combination with a prophylactic/therapeutic agent. Such antibodies can be useful for monitoring or prognosing the development or progression of a hypoproliferative disorder as part of a clinical testing procedure, such as determining the efficacy of a particular therapy. Such diagnosis and detection can accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴ Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (35S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

5.5 Identification of EphA2 Antagonistic Agents of the Invention

The invention provides methods of assaying and screening for EphA2 antagonistic agents of the invention by incubating agents with cells that express EphA2, particularly epithelial and/or endothelial cells, and then assaying for an ability to decrease EphA2 cytoplasmic tail phosphorylation, promotes EphA2 kinase activity (other than autophosphorylation or ligand-mediated EphA2 signaling), inhibit EphA2-endogenous ligand interaction, promote proliferation/growth/survival of EphA2-expressing cells, and/or increase maintenance/reconstitution cell layer integrity thereby identifying an EphA2 agent of the invention. The invention also encompasses the use of in vivo assays to identify EphA2 agents, e.g., by reduction in pathological symptoms in animal models of a hypoproliferative cell disorder or disorder involving increased cell death.

5.5.1 Antagonistic Agents That Decrease EphA2 Cytoplasmic Tail Phosphorylation

The invention provides methods of assaying and screening for EphA2 antagonistic agents that decrease EphA2 cytoplasmic tail phosphorylation. Such antagonistic agents of the invention decrease EphA2 internalization and degradation due to EphA2 cytoplasmic tail phosphorylation. Thus, EphA2 protein levels remain higher than they would otherwise in the absence of an antagonistic agent that decreases EphA2 cytoplasmic tail phosphorylation. In one embodiment, EphA2 antagonistic agents decrease EphA2 cytoplasmic tail phosphorylation. In another embodiment, EphA2 antagonistic agents decrease Eph A2 internalization and degradation. Any method known in the art to assay either the level of EphA2 phosphorylation or expression can be used to screen EphA2 agents to determine their ability to decrease EphA2 cytoplasmic tail phosphorylation or EphA2 degradation, e.g., immunoprecipitation, western blot, ELISAs, and phosphorylation assays (e.g., OMNI-PHOS™ kit available from Chemicon International, Temecula, Calif.). Ligand-mediated EphA2 cytoplasmic tail phosphorylation has been shown to cause the EphA2 cytoplasmic tail to interact with the PTB and SH2 domains of SHC, promote nuclear translocation and phosphorylation of ERK kinases, and increase nuclear induction of the Elk-1 transcription factor (Pratt and Kinch, 2002, Oncogene 21:7690-9). In another embodiment, EphA2 antagonistic agents decrease ligand-mediated EphA2 signaling. In a specific embodiment, EphA2 antagonistic agents decrease ligand-mediated EphA2 interaction with SHC. In another specific embodiment, EphA2 antagonistic agents decrease ligand-mediated nuclear translocation and/or phosphorylation of ERK kinases. In another specific embodiment, EphA2 antagonistic agents decrease ligand-mediated nuclear induction of the Elk-I transcription factor. Any method in the art to assay ligand-mediated EphA2 signaling can be used to screen EphA2 agents to determine their ability to decrease ligand-mediated EphA2 signaling, e.g., reporter gene assay, immunoprecipitation, immunoblotting, GST fusion protein pull down assay (see, e.g., Pratt and Kinch, 2002, Oncogene 21:7690-9).

5.5.2 Antagonistic Agents That Increase EphA2 Enzymatic Activity

The invention provides methods of assaying and screening for EphA2 antagonistic agents that increase the enzymatic activity of EphA2 (other than autophosphorylation or ligand-mediated EphA2 signaling). Such antagonistic agents are identified by assaying for the ability of a candidate EphA2 agent to increase the level of EphA2 enzymatic activity that is present in an EphA2-expressing cell, particularly an epithelial and/or endothelial cell, when unbound to ligand. In some embodiments, the candidate agents are screened for ability to increase EphA2 enzymatic activity (e.g., in a kinase activity assay) that is present when EphA2 is not bound to ligand. In other embodiments, candidate agents are screened for the ability to increase signaling through the EphA2 signaling cascade (e.g., in a reporter gene assay such as a CATalyse Reporter Gene Assay available from Serologicals Corporation, Norcross, Ga.) that is active when EphA2 is not bound to ligand.

5.5.3 Antagonistic Agents That Decrease EphA2-Endoienous Ligand Interaction

The invention provides methods of assaying and screening for EphA2 antagonistic agents that decrease or disrupt EphA2-endogenous ligand interaction. In one embodiment, the antagonistic agents (preferably one that possesses a structurally or functionally similar epitope as Ephrin A1) are screened for ability to competitively bind cellular EphA2 so it cannot bind with its natural ligand Ephrin A1. EphA2 binding to such a non-endogenous ligand preferably does not result in the type or degree of signaling that EphA2 binding its endogenous ligand elicits. In another embodiment, the antagonistic agents (preferably a soluble endogenous ligand binding extracellular domain of EphA2) are screened for ability to competitively bind Ephrin A1 so Ephrin A1 does not bind cellular EphA2. The number of antagonistic agents that competitively bind Ephrin A1 or cellular EphA2 can be analyzed by various known techniques including, but not limited to, ELISAs, immunoblots, radio-immunoprecipitations, etc. The invention provides compositions wherein the percentage binding between cellular EphA2 and its endogenous ligand Ephrin A1 is less than 99%, 95%, 90%, 80%, 70%, 60%, 50%, 40%, 30%, 20%, 10%, 5%, 1%.

5.5.4 Cell Proliferation Stimulative Agents

The invention provides methods of assaying and screening for EphA2 antagonistic agents of the invention that promote proliferation/growth/survival of EphA2-expressing cells, particularly epithelial and/or endothelial cells. Many assays well-known in the art can be used to assess survival, growth, and/or proliferation; for example, cell proliferation can be assayed by measuring (³H)-thymidine incorporation, by direct cell count, by detecting changes in transcription, translation or activity of known genes such as cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as western blotting or immunoprecipitation using commercially available antibodies (for example, many cell cycle marker antibodies are from Santa Cruz Inc.). mRNA can be quantitated by methods that are well known and routine in the art, for example by northern analysis, RNase protection, the polymerase chain reaction in connection with the reverse transcription, etc. Cell viability can be assessed by using trypan-blue staining or other cell death or viability markers known in the-art.

The present invention provides for cell cycle and cell proliferation analysis by a variety of techniques known in the art, including but not limited to the following:

As one example, bromodeoxyuridine (BRDU) incorporation may be used as an assay to identify proliferating cells. The BRDU assay identifies a cell population undergoing DNA synthesis by incorporation of BRDU into newly synthesized DNA. Newly synthesized DNA may then be detected using an anti-BRDU antibody (see Hoshino et al., 1986, Int. J. Cancer 38:369; Campana et al., 1988, J. Immunol. Meth. 107:79).

Cell proliferation may also be examined using (³H)-thymidine incorporation (see e.g., Chen, 1996, Oncogene 13:1395-403; Jeoung, 1995, J. Biol. Chem. 270:18367-73). This assay allows for quantitative characterization of S-phase DNA synthesis. In this assay, cells synthesizing DNA will incorporate (³H)-thymidine into newly synthesized DNA. Incorporation may then be measured by standard techniques in the art such as by counting of radioisotope in a Scintillation counter (e.g. Beckman L S 3800 Liquid Scintillation Counter).

Detection of proliferating cell nuclear antigen (PCNA) may also be used to measure cell proliferation. PCNA is a 36 kilodalton protein whose expression is elevated in proliferating cells, particularly in early G1 and S phases of the cell cycle and therefore may serve as a marker for proliferating cells. Positive cells are identified by immunostaining using an anti-PCNA antibody (see Li et al., 1996, Curr. Biol. 6:189-99; Vassilev et al., 1995, J. Cell Sci. 108:1205-15).

Cell proliferation may be measured by counting samples of a cell population over time (e.g. daily cell counts). Cells may be counted using a hemacytometer and light microscopy (e.g. HyLite hemacytometer, Hausser Scientific). Cell number may be plotted against time in order to obtain a growth curve for the population of interest. In a preferred embodiment, cells counted by this method are first mixed with the dye Trypan-blue (Sigma), such that living cells exclude the dye, and are counted as viable members of the population.

DNA content and/or mitotic index of the cells may be measured, for example, based on the DNA ploidy value of the cell. For example, cells in the GI phase of the cell cycle generally contain a 2N DNA ploidy value. Cells in which DNA has been replicated but have not progressed through mitosis (e.g. cells in S-phase) will exhibit a ploidy value higher than 2N and up to 4N DNA content. Ploidy value and cell-cycle kinetics may be further measured using propidum iodide assay (see e.g. Turner, et al., 1998, Prostate 34:175-81). Alternatively, the DNA ploidy may be determined by quantitation of DNA Feulgen staining (which binds to DNA in a stoichiometric manner) on a computerized microdensitometrystaining system (see e.g., Bacus, 1989, Am. J. Pathol.135:783-92). In an another embodiment, DNA content may be analyzed by preparation of a chromosomal spread (Zabalou, 1994, Hereditas.120:127-40; Pardue, 1994, Meth. Cell Biol. 44:333-351).

The expression of cell-cycle proteins (e.g., CycA. CycB, CycE, CycD, cdc2, Cdk4/6, Rb, p21, p27, etc.) provide crucial information relating to the proliferative state of a cell or population of cells. For example, identification in an anti-proliferation signaling pathway may be indicated by the induction of p21^{cip1}. Increased levels of p21 expression in cells results in delayed entry into G1 of the cell cycle (Harper et al., 1993, Cell 75:805-816; Li et al., 1996, Curr. Biol. 6:189-199). p21 induction may be identified by immunostaining using a specific anti-p21 antibody available commercially (e.g. Santa Cruz). Similarly, cell-cycle proteins may be examined by western blot analysis using commercially available antibodies. In another embodiment, cell populations are synchronized prior to detection of a cell cycle protein. Cell cycle proteins may also be detected by FACS (fluorescence-activated cell sorter) analysis using antibodies against the protein of interest.

EphA2 antagonistic agents of the invention can also be identified by their ability to change the length of the cell cycle or speed of cell cycle so that cell proliferation is decreased or inhibited. In one embodiment the length of the cell cycle is determined by the doubling time of a population of cells (e.g., using cells contacted or not contacted with one or more candidate EphA2 agents). In another embodiment, FACS analysis is used to analyze the phase of cell cycle progression, or purify GI, S, and G2/M fractions (see e.g., Delia et al., 1997, Oncogene 14:2137-47).

5.5.5 Antagonistic Agents That Increase Integrity of Cell Layer

The invention provides methods of assaying and screening for EphA2 antagonistic agents of the invention that increase the maintenance or reconstitution of the integrity of a cell layer, especially an epithelial and/or endothelial cell layer. Candidate agents are screened for their ability to maintain and/or reconstitute epithelial and/or endothelial cell layer integrity in a bicameral chamber (e.g., Boyden chamber, Ussing chamber, Tranwell chamber, etc.). For example, a bicameral chamber can be set up such that a monolayer of epithelial cells is present between an upper and lower well of medium. Cell layer integrity in the presence and absence of candidate EphA2 agents can be ascertained by a number of methods. For example, the degree of passive solute flow between chamber wells can be indicative of cell layer integrity. A marker molecule (e.g., stain, radioactive label) can be added to one of the wells and the time period it takes for the marker molecule to have access to the medium in the other well can be measured. Alternatively, the transepithelial electrical resistance may be measured to indicate the cell layer integrity. Increasing cell layer integrity is indicated by increasing transepithelial electrical resistance. See generally, Kim & Suh, 1993, Am. J. Physiol. 264:L308-15 and Nilsson et al., 1996, Eur. J. Endocrinol. 135:469-80.

5.6 Characterization and Demonstration of Therapeutic/Prophylactic Utility

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The anti-hypoproliferative cell disorder activity of the therapies used in accordance with the present invention also can be determined by using various experimental animal models for the study of hypoproliferative cell disorders or disorders involving increased cell death, especially those disorders relating to the destruction, shedding, or inadequate proliferation of epithelial and/or endothelial cells, particularly IC and lesions associated with IBD.

5.6.1 Demonstration of Therapeutic Utility

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which a patient tissue sample is grown in culture, and exposed to or otherwise administered a protocol, and the effect of such protocol upon the tissue sample is observed, e.g., decreased EphA2-endogenous ligand binding, upregulated EphA2 gene expression and/or translation, increases EphA2 protein stability or protein accumulation, decreased EphA2 cytoplasmic tail phosphorylation, increased proliferation of EphA2 expressing cells, increased survival of EphA2 expressing cells, and/or maintained/reconstituted integrity of an epithelial and/or endothelial cell layer. A demonstration of any of the aforementioned properties of the contacted cells indicates that the therapeutic agent is effective to treat the condition in the patient. Alternatively, instead of culturing cells from a patient, therapeutic agents and methods may be screened using cells of a epithelial and/or endothelial cell line. Many assays standard in the art can be used to assess such survival, growth, and/or proliferation; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining.

Compounds for use in therapy can be tested in suitable animal model systems prior to testing in humans, including but not limited to in rats, mice, chicken, cows, monkeys, rabbits, hamsters, etc. The compounds can then be used in the appropriate clinical trials.

Further, any assays known to those skilled in the art can be used to evaluate the prophylactic and/or therapeutic utility of the combinatorial therapies disclosed herein for treatment or prevention of a hypoproliferative cell disorder or disorder involving increased cell death. 5.6.2 Dosages

The amount of the composition of the invention which will be effective in the treatment, management, or prevention of hypoproliferative cell disorders or disorders involving increased cell deaths, especially those disorders relating to the destruction, shedding, or inadequate proliferation of epithelial and/or endothelial cells, particularly IC and lesions associated with IBD, can be determined by standard research techniques. For example, the dosage of the composition which will be effective in the treatment, management, or prevention of a hypoproliferative cell disorder or disorder involving increased cell death can be determined by administering the composition to an animal model such as, e.g., the animal models known to those skilled in the art. In addition, in vitro assays may optionally be employed to help identify optimal dosage ranges.

Selection of the preferred effective dose can be determined (e.g., via clinical trials) by a skilled artisan based upon the consideration of several factors which will be known to one of ordinary skill in the art. Such factors include the disorder to be treated or prevented, the symptoms involved, the patient's body mass, the patient's immune status and other factors known by the skilled artisan to reflect the accuracy of administered pharmaceutical compositions.

The precise dose to be employed in the formulation will also depend on the route of administration, and the seriousness of the hypoproliferative cell disorder or disorder involving increased cell death, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human and humanized antibodies have a longer half-life within the human body than antibodies from other species due to the immune response to the foreign polypeptides. Thus, lower dosages of human antibodies and less frequent administration is often possible.

For other therapeutic agents administered to a patient, the typical doses of various immunomodulatory or anti-UTI therapeutics are known in the art. Given the invention, certain preferred embodiments will encompass the administration of lower dosages in combination treatment regimens than dosages recommended for the administration of single agents.

The invention provides for any method of administrating lower doses of known prophylactic or therapeutic agents than previously thought to be effective for the prevention, treatment, management, or prevention of hypoproliferative cell disorders or disorders involving increased cell death, especially those disorders relating to the destruction, shedding, or inadequate proliferation of epithelial and/or endothelial cells, particularly IC and lesions associated with IBD. Preferably, lower doses of known immunomodulatory and anti-UTI agents are administered in combination with lower doses of EphA2 antagonistic agents of the invention. 5.7 Pharmaceutical Compositions

The compositions of the invention include bulk drug which is useful in the manufacture of oral pharmaceutical compositions (e.g., non-sterile compositions) and parenteral pharmaceutical compositions (i.e., compositions that are suitable for administration to a subject or patient which are sterile) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of one or more EphA2 antagonistic agents of the invention and a pharmaceutically acceptable carrier or an agent that increases EphA2 expression and a pharmaceutically acceptable carrier. In a further embodiment, the composition of the invention further comprises an additional therapeutic, e.g., immunomodulatory or anti-UTI agent.

In a specific embodiment, the term “pharmaceutically acceptable” means approved by a regulatory agency of the Federal or a state goverunent or listed in the U.S. Pharmacopeia or other generally recognized pharmacopeia for use in animals, and more particularly in humans. The term “carrier” refers to a diluent, excipient adjuvant (e.g., Freund's adjuvant or, more preferably, MF59C.1 adjuvant available from Chiron, Emeryville, Calif.), excipient, or vehicle with which the therapeutic is administered. Other such adjuvants may include, but are not limited to mineral gels such as aluminum hydroxide; surface active substances such as lysolecithin, pluronic polyols, polyanions; other peptides; oil emulsions; and potentially useful human adjuvants such as BCG and Corynebacterium parvum. The pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known and can be used to administer an EphA2 antagonistic agent of the invention or the combination of an EphA2 antagonistic agent of the invention and a non-EphA2-based prophylactic /therapeutic agent useful for preventing/treating a hypoproliferative cell disorders or disorders involving increased cell death, especially those disorders relating to the destruction, shedding, or inadequate proliferation of epithelial and/or endothelial cells, particularly IC and lesions associated with IBD, e.g., encapsulation in liposomes, microparticles, microcapsules, recombinant cells capable of expressing the antibody or antibody fragment, receptor-mediated endocytosis (see, e.g., Wu and Wu, 1987, J. Biol. Chem. 262:4429-4432), construction of a nucleic acid as part of a retroviral or other vector, etc. Methods of administering a prophylactic or therapeutic agent of the invention include, but are not limited to, parenteral administration (e.g., intradermal, intramuscular, intraperitoneal, intravenous and subcutaneous), epidural, and mucosal (e.g., intranasal, inhaled, and oral routes). In a specific embodiment, prophylactic or therapeutic agents of the invention are administered intramuscularly, intravenously, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the prophylactic or therapeutic agent can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; International Patent Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Patent Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

Pharmaceutical compositions for use in accordance with the present invention may be formulated in conventional manner using one or more physiologically acceptable carriers or excipients. Preferably, agents are formulated and administered systemically. Techniques for formulation and administration may be found in “Remington: The Science and Practice of Pharmacy”, 19th ed., 1995, Lippincott Williams & Wilkins, Baltimore, Md.

Thus, the EphA2 antagonistic agents of the invention (e.g., EphA2 cytoplasmic tail phosphorylation inhibitors, EphA2-ligand interaction inhibitors, EphA2 enzymatic activity (other than autophosphorylation or ligand-mediated EphA2 signaling) promoters, and cell proliferation stimulative agents) and their physiologically acceptable salts and solvates may be formulated for administration by inhalation or insufflation (either through the mouth or the nose) or oral, parenteral or mucosal (such as buccal, vaginal, rectal, sublingual) administration. In a preferred embodiment, local or systemic parenteral administration is used.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the prophylactic or therapeutic agents for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The prophylactic or therapeutic agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The prophylactic or therapeutic agents may also be formulated in rectal compositions such as suppositories or retention enemas, e.g., containing conventional suppository bases such as cocoa butter or other glycerides.

In addition to the formulations described previously, the prophylactic or therapeutic agents may also be formulated as a depot preparation. Such long acting formulations may be administered by implantation (for example subcutaneously or intramuscularly) or by intramuscular injection. Thus, for example, the prophylactic or therapeutic agents may be formulated with suitable polymeric or hydrophobic materials (for example as an emulsion in an acceptable oil) or ion exchange resins, or as sparingly soluble derivatives, for example, as a sparingly soluble salt.

The invention also provides that a prophylactic or therapeutic agent is packaged in a hermetically sealed container such as an ampoule or sachette indicating the quantity. In one embodiment, the prophylactic or therapeutic agent is supplied as a dry sterilized lyophilized powder or water free concentrate in a hermetically sealed container and can be reconstituted, e.g., with water or saline to the appropriate concentration for administration to a subject.

In a preferred embodiment of the invention, the formulation and administration of various chemotherapeutic, biological/immunotherapeutic and hormonal therapeutic agents are known in the art and often described in the Physician 's Desk Reference, 56^{th} ed. (2002).

In other embodiments of the invention, radiation therapy agents such as radioactive isotopes can be given orally as liquids in capsules or as a drink. Radioactive isotopes can also be formulated for intravenous injections. The skilled oncologist can determine the preferred formulation and route of administration.

In certain embodiments the antagonistic monoclonal antibodies of the invention, are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

5.7.1. Gene Therapy

In specific embodiments, antagonistic agents of the invention that are nucleotides are administered to treat, manage, or prevent a hypoproliferative cell disorder or disorder involving increased cell death, especially those disorders relating to the destruction, shedding, or inadequate proliferation of epithelial and/or endothelial cells, particularly IC and lesions associated with IBD, by way of gene therapy. Gene therapy refers to therapy performed by the administration to a subject of an expressed or expressible nucleic acid. In this embodiment of the invention, the antisense nucleic acids are produce and mediate a prophylactic or therapeutic effect.

Any of the methods for gene therapy available in the art can be used according to the present invention. Exemplary methods are described below.

For general reviews of the methods of gene therapy, see Goldspiel et al., 1993, Clinical Pharmacy 12:488; Wu and Wu, 1991, Biotherapy 3:87; Tolstoshev, 1993, Ann. Rev. Pharmacol. Toxicol. 32:573; Mulligan, 1993, Science 260:926-932; and Morgan and Anderson, 1993, Ann. Rev. Biochem. 62:191; May, 1993, TIBTECH 11:155. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises Ephrin A1 nucleic acids that decrease Ephrin A1 expression, said nucleic acids being part of an expression vector that expresses the nucleic acid in a suitable host. In particular, such nucleic acids have promoters, preferably heterologous promoters, said promoter being inducible or constitutive, and, optionally, tissue-specific. In another particular embodiment, nucleic acid molecules are used in which the nucleic acid that decrease Ephrin A1 expression and any other desired sequences are flanked by regions that promote homologous recombination at a desired site in the genome, thus providing for intrachromosomal expression of the nucleic acids that decrease Ephrin A1 expression (Koller and Smithies, 1989, PNSA 86:8932; Zijlstra et al., 1989, Nature 342:435).

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy. In a specific embodiment, the nucleic acid sequences are directly administered in vivo. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429) (which can be used to target cell types specifically expressing the receptors), etc. In another embodiment, nucleic acid-ligand complexes can be formed in which the ligand comprises a flisogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., International Patent Publication Nos. WO 92/06180; WO 92/22635; W092/203 16; W093/14188, WO 93/20221). Alternatively, the nucleic acid can be introduced intracellularly and incorporated within host cell DNA for expression, by homologous recombination (Koller and Smithies, 1989, PNSA 86:8932; and Zijlstra et al., 1989, Nature 342:435).

In a specific embodiment, viral vectors that contain the nucleic acid sequences that decrease Ephrin A1 expression are used. For example, a retroviral vector can be used (see Miller et al., 1993, Meth. Enzymol. 217:581). These retroviral vectors contain the components necessary for the correct packaging of the viral genome and integration into the host cell DNA. The nucleic acid sequences to be used in gene therapy are cloned into one or more vectors, which facilitates delivery of the nucleic acid into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Adenoviruses have the advantage of being capable of infecting non-dividing cells. Kozarsky and Wilson, 1993, Current Opinion in Genetics Development 3:499 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431; Rosenfeld et al., 1992, Cell 68:143; Mastrangeli et al., 1993, J. Clin. Invest. 91:225; International Patent Publication No. W094/12649; and Wang et al., 1995, Gene Therapy 2:775. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217:599; Cohen et al., 1993, Meth. Enzymol. 217:618) and maybe used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. The amount of cells envisioned for use depends on the desired effect, patient state, etc., and can be determined by one skilled in the art.

5.8 Kits

The invention provides a pharmaceutical pack or kit comprising one or more containers filled with an EphA2 antagonistic agent of the invention. Additionally, one or more other prophylactic or therapeutic agents useful for the treatment of a hypoproliferative epithelial and/or endothelial cell disorder or other relevant agents can also be included in the pharmaceutical pack or kit. In certain embodiments, the other prophylactic or therapeutic agent is an immunomodulatory agent (e.g., anti-IL-9 antibody). In other embodiments, the prophylactic or therapeutic agent is an anti-UTI agent (e.g., anti-FimH antibody). The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

6. Equivalents

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

1. A method of treating a hypoproliferative cell disorder or disorder involving increased cell death in a patient in need thereof, said method comprising administering to said patient a therapeutically effective amount of an EphA2 antagonistic agent.

2. The method of claim 1 wherein said hypoproliferative cell disorder or disorder involving increased cell death comprises the destruction, shedding, or inadequate proliferation of epithelial cells.

3. The method of claim 2 wherein said hypoproliferative cell disorder is interstitial cystitis or a lesion associated with inflammatory bowel disease.

4. The method of claim 1 wherein said hypoproliferative cell disorder or disorder involving increased cell death comprises the destruction, shedding, or inadequate proliferation of endothelial cells.

5. The method of claim 1 wherein said administration increases the proliferation or survival of an epithelial cell relative to the level of proliferation or survival in an untreated epithelial and/or endothelial cell.

6. The method of claim 1 wherein said administration increases the proliferation or survival of an endothelial cell relative to the level of proliferation or survival in an untreated epithelial and/or endothelial cell.

7. The method of claim 1 wherein said administration decreases EphA2 cytoplasmic tail phosphorylation relative to the untreated level of EphA2 cytoplasmic tail phosphorylation.

8. The method of claim 1 wherein said administration increases the integrity of an epithelial cell layer relative to the level of integrity of an untreated epithelial cell layer.

9. The method of claim 1 wherein said administration increases the integrity of an endothelial cell layer relative to the level of integrity of an untreated endothelial cell layer.

10. The method of claim 1 wherein said administration increases EphA2 gene expression or translation.

11. The method of claim 1 wherein said EphA2 antagonistic agent is an EphA2 polypeptide fragment comprising a ligand binding domain of EphA2.

12. The method of claim 1 wherein said EphA2 antagonistic agent is an antibody or antigen binding fragment thereof.

13. The method of claim 12 wherein said EphA2 antagonistic agent is an EphrinA1 antibody or antigen binding fragment thereof.

14. The method of claim 12 wherein the said antibody is a monoclonal antibody.

15. The method of claim 14 wherein said monoclonal antibody is a human antibody.

16. The method of claim 14 wherein said monoclonal antibody is humanized.

17. The method of claim 1 wherein said EphA2 antagonistic agent is chosen from the group consisting of a small molecule antagonist, enzymatic activity antagonist, EphrinA1 siRNA or eiRNA molecule, and EphrinA1 antisense molecule.

18. The method of claim 1 wherein said antagonistic agent increases EphA2 protein stability or protein accumulation.

19. The method of claim 1 wherein said administration decreases EphA2-endogenous ligand binding relative to the amount of untreated EphA2-endogenous ligand binding.

20. The method of claim 19 wherein said endogenous ligand is Ephrin A1.

21. The method of claim 1 further comprising the administration of one or more additional hypoproliferative cell disorder therapies that do not alter EphA2 expression or activity.

22. The method of claim 21 wherein said additional hypoproliferative cell disorder therapies consist of an immunomodulatory agent or an anti-urinary tract infection agent.

23. The method of claim 22 wherein said immunomodulatory agent is an antibody that immunospecifically binds IL-9.
